(19) United States
(12) Patent Application Publication
(10) Pub. No.: US 2008/0262056 A1
(43) Pub. Date: Oct. 23, 2008

(54) Oxindole Oxazolidinones as Antibacterial Agents

(75) Inventors: Gary Wayne Luehr, Hayward, CA (US); Rama Jain, Fremont, CA (US); Adam Robert Renslo, Oakland, CA (US); Mikhail Fedorovich Gordeev, Castro Valley, CA (US); Vara Prasad Venkata Nagendra Josyula, Superior Township, MI (US)

Correspondence Address: PFIZER INC;Steve T. Zelson, 150 EAST 42ND STREET, 5TH FLOOR - STOP 49, NEW YORK, NY, 10017-5612, US

(73) Assignee: Pfizer Inc

(21) Appl. No.: 11/573,161

(22) PCT Filed: Jul. 19, 2005
(86) PCT No.: PCT/IB2005/002196

§ 371 Date: May 15, 2008

Related U.S. Application Data
(60) Provisional application No. 60/599,822, filed on Aug. 6, 2004.

(51) Int. Cl. A61K 31/422; C07D 413/10; A61P 31/04

(52) U.S. Cl. 514/376; 548/231

(57) ABSTRACT

The present invention relates to novel oxazolidinones derivatives of oxindoles of formula I or a pharmaceutically acceptable salt thereof wherein: Y¹ is CH or CF; R¹ is —C₁₋₄alkyl, optionally substituted with a fluoro atom, or R¹ is —C₃₋₅cycloalkyl; and R² is —C₁₋₂alkyl. These compounds are useful as antibacterial agents.

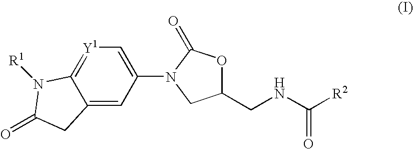

### FIELD OF INVENTION

The present invention relates to novel oxazolidinones derivatives of oxindoles, pharmaceutical compositions thereof, methods for their use, and methods for preparing these compounds. These compounds have potent activities against against gram-positive and/or gram-negative bacteria.

### BACKGROUND OF THE INVENTION

Due to ever-increasing antibiotic resistance, structurally novel antibacterials with a new mode of action have become increasingly important in the treatment of bacterial infections. Effective antibacterials exhibit potent activity against a number of human and veterinary pathogens, including gram-positive aerobic bacteria such as multiple resistant staphylococci and streptococci, anaerobic organisms such as bacteroides and clostridia species, and acid-fast organisms such as Mycobacterium tuberculosis and Mycobacterium avium.

Among newer antibacterial agents, oxazolidinone compounds are the most recent synthetic class of antimicrobials active against a number of pathogenic microorganisms. This invention provides novel oxindole derivatives of oxazolidinones, and their preparation.

### INFORMATION DISCLOSURE

WO 200281470 discloses oxazolidinone compounds useful for treating bacterial infection.

WO 200032599 discloses oxazolidinone derivatives useful for treatment of microbial infections.

WO 200029396 discloses 3-phenyl-5-aminomethyl-oxazolidinone derivatives useful as antibacterial agents.

WO 9937630 discloses oxazolidinone derivatives including combinatorial libraries.

WO 9737981 discloses oxazolidinones

DE 19604223 discloses new substituted oxazolidinone compounds useful as antibacterial agents against.

DE 19649095 discloses 5-(acyl-aminomethyl)-3-hetero-aryl-oxazolidinone compounds useful as antibacterial agents.

EP 694543 discloses hetero-aryl substd. oxazolidinone derivatives useful as antibacterial agents.

EP 693491 discloses 3-hetero-aryl-2-oxazolidinone derivatives useful as antibacterial agents.

EP 609905 discloses indaxolyl, benzimidazolyl, and benzofrizxolyl oxazolidinone derivatives useful as antibacterial agents.

U.S. Pat. No. 5,164,510 discloses 5-Indolinylioxazolidin-2-one(s) useful as antibacterial agents.

US2002016323 dusckises oxazolidinone antibacterial agents.

US 2002032348 discloses process to prepare oxazolidinones.

US2002143009 discloses bicyclic oxazolidinone derivatives useful as antimicrobial agents.

US 2003/216330 discloses parenteral, intravenous, and oral administration of oxazolidinones for treating diabetic foot infections.

US 2004/176610 discloses antibacterial indolone oxazolidinone as antibacterial agents.

US2004147760 discloses N-aryl-2-oxazolidinone-5-carboxamides having antibacterial activity useful for treating microbial infections.

### SUMMARY OF THE INVENTION

The present invention provides a compound of formula I

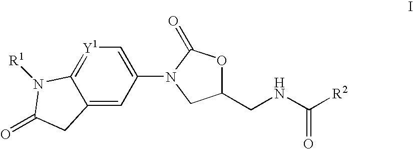

or a pharmaceutically acceptable salt thereof wherein:

### Y¹ is CH or CF;

R¹ is —C₁₋₄alkyl, optionally substituted with a fluoro atom, or R¹ is
—C₃₋₅cycloalkyl; and
R² is —C₁₋₂alkyl.

In another aspect, the present invention also provides:

a pharmaceutical composition which comprises a pharmaceutically acceptable carrier and an effective amount of a compound of formula I,

a method for treating gram-positive or gram-negative microbial infections in a mammal by administering to the subject in need a therapeutically effective amount of a compound of formula I or a pharmaceutically acceptable salt thereof, and

a use of a compound of formula I or a pharmaceutically acceptable salt thereof to prepare a medicament for treating gram-positive or gram-negative microbial infections.

The invention may also provide novel intermediates and novel processes that are useful for preparing compounds of formula I.

### DETAILED DESCRIPTION OF THE INVENTION

Unless otherwise stated, the following terms used in the specification and claims have the meanings given below:

The carbon atom content of various hydrocarbon-containing moieties is indicated by a prefix designating the minimum and maximum number of carbon atoms in the moiety, i.e., the prefix Cᵢ₋ⱼ indicates a moiety of the integer “i” to the integer “j” carbon atoms, inclusive. Thus, for example, C₁₋₆ alkyl refers to alkyl of one to seven carbon atoms, inclusive.

The term alkyl refesr to both straight and branched groups, but reference to an individual radical such as “propyl” embraces only the straight chain radical, a branched chain isomer such as “isopropyl” being specifically referred to.

The term “C₃₋₅cycloalkyl” refers to a cyclic saturated monovalent hydrocarbon group of three to five carbon atoms, e.g., cyclopropyl, and the like.

The term “halo” refers to fluoro (F), chloro (Cl), bromo (Br), or iodo (I). The term “a pharmaceutically acceptable salt” of a compound means a salt that is pharmaceutically acceptable and that possesses the desired pharmacological activity of the parent compound.

The term “pharmaceutically acceptable carrier” means a carrier that is useful in preparing a pharmaceutical composition that is generally safe, non-toxic and neither biologically nor otherwise undesirable, and includes a carrier that is acceptable for veterinary use as well as human pharmaceutical use. “A pharmaceutically acceptable carrier” as used in the specification and claims includes both one and more than one such carrier.

The term “mammal” refers to human or warm-blooded animals including livestock and companion animals.

The term “optional” or “optionally” means that the subsequently described event or circumstance may, but need not, occur, and that the description includes instances where the event or circumstance occurs and instances in which it does not.

Compounds that have the same molecular formula but differ in the nature or sequence of bonding of their atoms or the arrangement of their atoms in space are termed “isomers”. Isomers that differ in the arrangement of their atoms in space are termed “stereoisomers”.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, tautomeric, or stereoisomeric form, or mixture thereof, of a compound of the invention, which possesses the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine antiviral activity using the standard tests described herein, or using other similar tests which are well known in the art.

The term “treating” or “treatment” of a disease includes: (1) preventing the disease, i.e. causing the clinical symptoms of the disease not to develop in a mammal that may be exposed to or predisposed to the disease but does not yet experience or display symptoms of the disease; (2) inhibiting the disease, i.e., arresting or reducing the development of the disease or its clinical symptoms; or (3) relieving the disease, i.e., causing regression of the disease or its clinical symptoms.

The term “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

The term “leaving group” has the meaning conventionally associated with it in synthetic organic chemistry i.e., an atom or group capable of being displaced by a nucleophile and includes halogen, alkylsulfonyloxy, ester, or amino such as chloro, bromo, iodo, mesyloxy, tosyloxy, trifluorosulfonyloxy, methoxy, N,O-dimethylhydroxyl-amino, and the like.

The compounds of the present invention are generally named according to the IUPAC or CAS nomenclature system.

Abbreviations which are well known to one of ordinary skill in the art may be used (e.g. “Ph” for phenyl, “Me” for methyl, “Et” for ethyl, “h” for an hour or hours and “rt” for room temperature).

Specific and preferred values listed below for radicals, substituents, and ranges, are for illustration only; they do not exclude other defined values or other values within defined ranges for the radicals and substituents.

Specifically, alkyl denotes both straight and branched groups; but reference to an individual radical such as “propyl” embraces only the straight chain radical, a branched chain isomer such as “isopropyl” being specifically referred to.

Specifically, alkyl is methyl, ethyl, propyl, isopropyl, butyl, iso-butyl, sec-butyl, and their isomeric forms thereof.

Specifically, cycloalkyl is cyclopropyl, cyclobutyl, cyclopentyl, and their isomeric forms thereof.

Specifically, halo is fluoro (F), or chloro (Cl).

Specifically, Y¹ is CH.

Specifically, R¹ is C₁₋₃alkyl.

Specifically, R¹ is methyl, or isopropyl.

Specifically, R² is methyl.

Examples of the present invention are:
(1) (S)—N-[3-(1-methyl-2-oxo-2,3-dihydro-1H-indol-5-yl)-2-oxo-oxazolidin-5-ylmethyl]-acetamide,
(2) (S)—N-[3-(1-methyl-2-oxo-2,3-dihydro-1H-indol-5-yl)-2-oxo-oxazolidin-5-ylmethyl]-propionamide,
(3) N-[3-(7-fluoro-1-methyl-2-oxo-2,3-dihydro-1H-indol-5-yl)-2-oxo-oxazolidin-5-ylmethyl]-acetamide,
(4) (S)—N-[3-(7-fluoro-1-methyl-2-oxo-2,3-dihydro-1H-indol-5-yl)-2-oxo-oxazolidin-5-ylmethyl]-propionamide,
(5) (S)—N-[3-(1-ethyl-2-oxo-2,3-dihydro-1H-indol-5-yl)-2-oxo-oxazolidin-5-ylmethyl]-acetamide,
(6) (S)—N-[3-(1-ethyl-2-oxo-2,3-dihydro-1H-indol-5-yl)-2-oxo-oxazolidin-5-ylmethyl]-propionamide,
(7) (S)—N-[3-(1-ethyl-7-fluoro-2-oxo-2,3-dihydro-1H-indol-5-yl)-2-oxo-oxazolidin-5-ylmethyl]-acetamide,
(8) (S)—N-[3-(1-ethyl-7-fluoro-2-oxo-2,3-dihydro-1H-indol-5-yl)-2-oxo-oxazolidin-5-ylmethyl]-propionamide,
(9) (S)—N-{3-[1-(2-fluoro-ethyl)-2-oxo-2,3-dihydro-1H-indol-5-yl]-2-oxo-oxazolidin-5-ylmethyl}-acetamide,
(10) (S)—N-{3-[1-(2-fluoro-ethyl)-2-oxo-2,3-dihydro-1H-indol-5-yl]-2-oxo-oxazolidin-5-ylmethyl}-propionamide,
(11) (S)—N-{3-[7-fluoro-1-(2-fluoro-ethyl)-2-oxo-2,3-dihydro-1H-indol-5-yl]-2-oxo-oxazolidin-5-ylmethyl}-acetamide,
(12) (S)—N-{3-[7-fluoro-1-(2-fluoro-ethyl)-2-oxo-2,3-dihydro-1H-indol-5-yl]-2-oxo-oxazolidin-5-ylmethyl}-propionamide,
(13) (S)—N-[3-(1-isopropyl-2-oxo-2,3-dihydro-1H-indol-5-yl)-2-oxo-oxazolidin-5-ylmethyl]-acetamide
(14) (5)-N-[3-(1-isopropyl-2-oxo-2,3-dihydro-1H-indol-5-yl)-2-oxo-oxazolidin-5-ylmethyl]-propionamide,
(15) (S)—N-[3-(7-fluoro-1-isopropyl-2-oxo-2,3-dihydro-1H-indol-5-yl)-2-oxo-oxazolidin-5-ylmethyl]-acetamide,
(16) (S)—N-[3-(7-fluoro-1-isopropyl-2-oxo-2,3-dihydro-1H-indol-5-yl)-2-oxo-oxazolidin-5-ylmethyl]-propionamide,
(17) (S)—N-[2-oxo-3-(2-oxo-1-propyl-2,3-dihydro-1H-indol-5-yl)-oxazolidin-5-ylmethyl]-acetamide,
(18) (S)—N-[2-oxo-3-(2-oxo-1-propyl-2,3-dihydro-1H-indol-5-yl)-oxazolidin-5-ylmethyl]-propionamide,
(19) (S)—N-[3-(7-fluoro-2-oxo-1-propyl-2,3-dihydro-1H-indol-5-yl)-2-oxo-oxazolidin-5-ylmethyl]-acetamide,
(20) (S)—N-[3-(7-Fluoro-2-oxo-1-propyl-2,3-dihydro-1H-indol-5-yl)-2-oxo-oxazolidin-5-ylmethyl]-propionamide,
(21) (S)—N-[3-(1-cyclopropyl-2-oxo-2,3-dihydro-1H-indol-5-yl)-2-oxo-oxazolidin-5-ylmethyl]-acetamide,
(22) (S)—N-[3-(1-cyclopropyl-2-oxo-2,3-dihydro-1H-indol-5-yl)-2-oxo-oxazolidin-5-ylmethyl]-propionamide,
(23) (S)—N-[3-(1-cyclopropyl-7-fluoro-2-oxo-2,3-dihydro-1H-indol-5-yl)-2-oxo-oxazolidin-5-ylmethyl]-acetamide,
(24) (S)—N-[3-(1-sec-butyl-2-oxo-2,3-dihydro-1H-indol-5-yl)-2-oxo-oxazolidin-5-ylmethyl]-acetamide,
(25) (S)—N-[3-(1-sec-butyl-2-oxo-2,3-dihydro-1H-indol-5-yl)-2-oxo-oxazolidin-5-ylmethyl]-propionamide,
(26) (S)—N-[3-(1-cyclopropylmethyl-2-oxo-2,3-dihydro-1H-indol-5-yl)-2-oxo-oxazolidin-5-ylmethyl]-acetamide,
(27) (S)—N-[3-(1-cyclopropylmethyl-2-oxo-2,3-dihydro-1H-indol-5-yl)-2-oxo-oxazolidin-5-ylmethyl]-propionamide,
(28) (S)—N-{3-[1-(2-fluoro-1-methyl-ethyl)-2-oxo-2,3-dihydro-1H-indol-5-yl]-2-oxo-oxazolidin-5-ylmethyl}-acetamide, or
(29) (S)—N-[3-(1-cyclopropylmethyl-7-fluoro-2-oxo-2,3-dihydro-1H-indol-5-yl)-2-oxo-oxazolidin-5-ylmethyl]-acetamide.

Compounds of this invention can be prepared in accordance with one or more of the Schemes discussed below. All of the starting materials may be prepared by the following descriptions, by procedures that would be well known to one of ordinary skill in organic chemistry, or are commercially available. Unless otherwise defined below, variables used in the Schemes are as defined in the specification or in the claims.

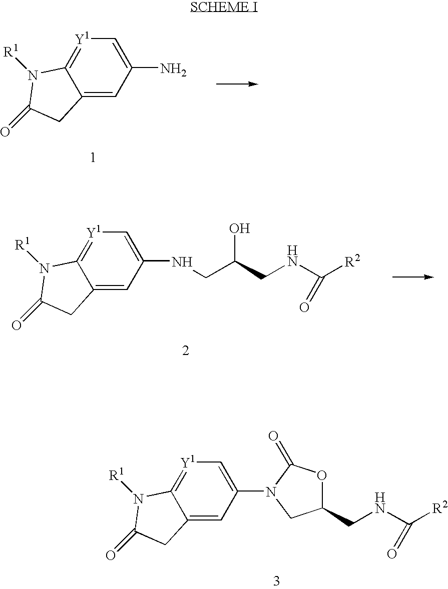

As shown in Scheme I, the substituted 5-amino-1,3-dihydroindol-2-one 1 is reacted with an appropriately protected (S)-glycidylamine and a Lewis acid such as lithium triflate in a suitable solvent such as acetonitrile at a suitable temperature, typically in the range from 20° C. to 110° C. depending on the solvent. Acetyl or Boc protected (S)-glycidylamines are conveniently prepared following methods described in International Patent Publication No. WO 02/32857. The amino alcohol 2 can then be ring closed to give the aryl oxazolidinones 3 using methods known to one skilled in the art. For instance, treatment of structures 2 with 1,1′-carbonyldiimidazole in a solvent such as acetonitrile or tetrahydrofuran at an appropriate temperature, typically in a range of 20° C. to 60° C., or with phosgene in a solvent such as toluene or methylene chloride, or mixtures thereof, in the presence of a base such as triethylamine at an appropriate temperature, typically in a range from −10° C. to 25° C., affords the oxazolidinone 3. If R² is a tert-butyl, the substrate may be further deprotected to the corresponding amine by treatment with an acid such as hydrochloric acid or trifluoroacetic acid in a suitable solvent such as methylene chloride, and then further converted to a urea (R⁵=NH₂ or NH-alkyl) or amide (R⁵=alkyl) 3 by treatment with an appropriate isocyanate or acylating reagent such as an acid chloride or anhydride in the presence of a suitable base such as triethylamine or pyridine.

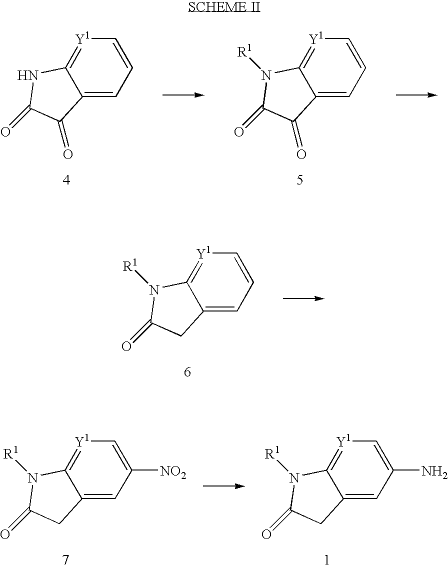

Oxindole intermediates may be prepared according to the method of Scheme II. Isatin 4, obtained commercially or conveniently prepared according to the methods of Gassman described in J. Org. Chem. 1977, 42, 1344 and U.S. Pat. Nos. 4,188,325 and 4,252,723, is treated with an alkylating agent, e.g., iodomethane, iodoethane, or iodopropane, in the presence of a suitable base (e.g. an amine base such as triethylamine or di-iso-propylethylamine or lithium, sodium, potassium or cesium carbonate) in a suitable organic solvent (e.g. DMF, THF, DMSO, dioxane or acetonitrile) at a temperature between 0° C. and 65° C. to afford N-alkylated isatin 5. Isatins 5 may be reduced to 1,3-dihydroindol-2-ones 6 by using red phosphorous and iodic acid, by use of hydrogen sulfide in pyridine/co-solvent mixture, or by the Wolf-Kishner reaction. The most convenient procedure involves heating isatin 5 in neat hydrazine hydrate at reflux in the absence of any additional base. 1,3-Dihydroindol-2-one 6 is nitrated regioselectively using methods known to one skilled in the art (e.g., nitric acid in concentrated sulfuric acid or acetic acid, or sodium nitrate in trifluoroacetic acid at temperatures between −20° C. and 25° C.). 5-Nitrooxindole 7 is then reduced by dissolving metal reduction (e.g., iron and ammonium chloride in ethanol/water) or catalytic hydrogenation to provide the 5-aminooxindole 1.

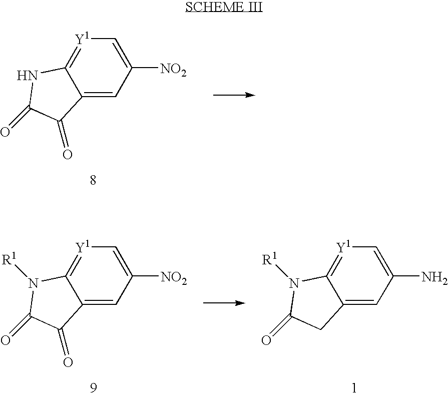

Alternatively, commercially available 5-nitroisatin 8 is treated with an appropriate alkylating agent, e.g., iodomethane, iodoethane, or iodopropane, in the presence of a suitable base base (e.g. an amine base such as triethylamine or di-iso-propylethylamine or lithium, sodium, potassium or cesium carbonate) in a suitable organic solvent (e.g. DMF, THF, DMSO, dioxane or acetonitrile) at a temperature between 0° C. and 65° C. to afford N-alkylated isatin 9. Isatin 9 may be reduced in one step to the requisite 5-aminooxindole 1 by heating in neat hydrazine hydrate at reflux temperatures or by catalytic hydrogenation.

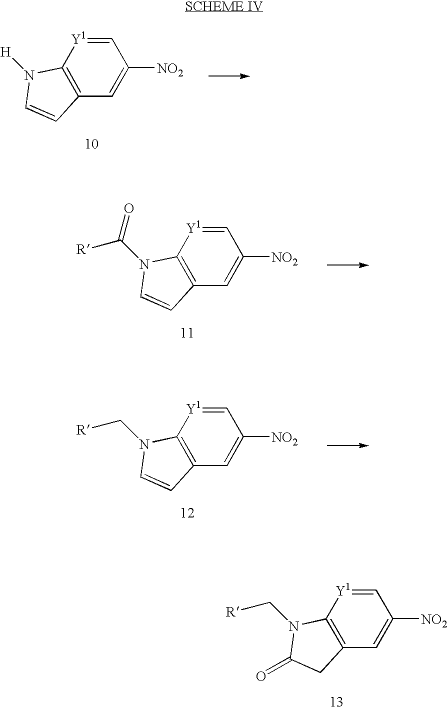

Scheme IV exemplifies another route to prepare 5-nitrooxindole. Commercially available 5-nitrooxindole 10 is acylated with an appropriate acid chloride or anhydride in the presence of a suitable base such as triethylamine or pyridine and in a suitable solvent such as methylene chloride at temperatures between 0° C. and 25° C. The resulting N-acylated oxindole 11 can be reduced to N-alkylindole 12 in high yields by BH₃.THF. N-Alkylindole 12 is further oxidized to the requisite 5-nitrooxindole 13 by a variety of known methods (e.g. DMSO/HCl, NBS).

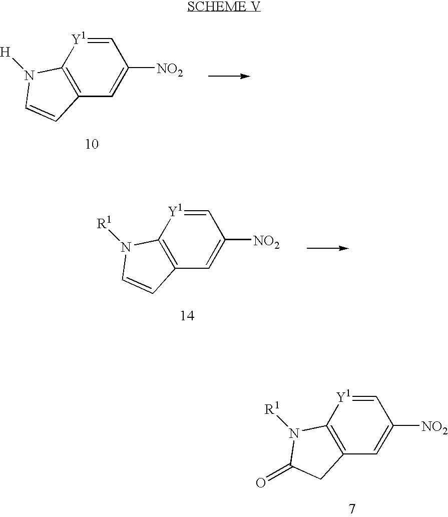

Alternatively, commercially available 5-nitrooxindole is treated with an appropriate alkylating agent, e.g., iodomethane, iodoethane, or iodopropane, in the presence of a suitable base (e.g., sodium hydride or lithium hexamethyldisilazane) in a suitable organic solvent (e.g. DMF, THF, or DMSO) at a temperature between 0° C. and 65° C. to afford N-alkylated indole 14. Indole 14 is oxidized to the requisite oxindole as discussed in Scheme IV.

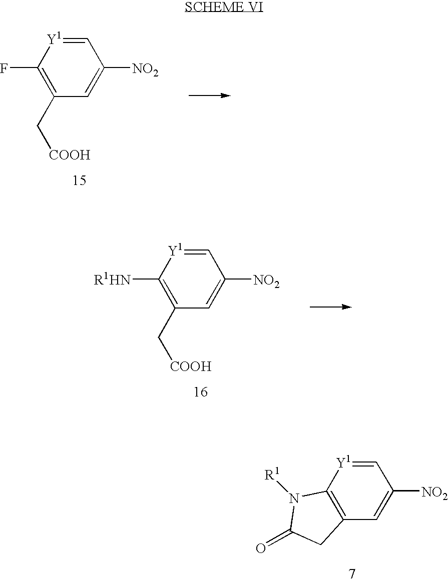

In another route exemplified by Scheme VI, an appropriately substituted 2-halo-5-nitrophenylacetic acid 15 (e.g., preferably 2-fluoro-5-nitrophenylacetic acid) is treated with ammonia or an optionally substituted amine (RNH₂) in a suitable solvent such as DMSO or acetonitrile at temperatures between 35° C. and 85° C. to afford aniline 16 (R═H or optionally substituted alkyl). Aniline 16 is treated with a strong acid such as HCl, H₂SO₄, or TFA to effect cyclization to the requisite 5-nitrooxindole 7.

### Medical and Veterinary Uses

It is known that as a chemical compound class, oxazolidinones generically inhibit monoamine oxidase (MAO), the enzyme responsible for preventing acute blood pressure elevation by the endogenous and dietary amine, tyramine. Accordingly, there is a demand to discover oxazolidinone antibiotics, which possess minimum MAO inhibitory activity to lower risk of potential drug-drug interactions. It has been discovered that, compounds of the present invention has unexceptedly weak MAO inhibitory activity, which indicates it possess the capacity to minimize or eliminate potential drug-drug interactions since strong inhibition of monoamine oxidase can result in altered clearance rates for other compounds normally metabolized by it, including several pharmaceuticals.

The compounds of the present invention may be used for the treatment of infectious, Gram-positive bacterial infections caused by a variety of bacterial organisms, including those that require long-term therapy (>28 days).

Examples of the bacterial organisms include gram-positive bacteria such as multiple resistant staphylococci, for example S. aureus and S. epidennidis; multiple resistant streptococci, for example S. pneumoniae and S. pyogenes; and multiple resistant Enterococci, for example E. faecalis; gram negative aerobic bacteria such as Haemophilus, for example H. influenzae and Moraxella, for example M. catarrhalis; as well as anaerobic organisms such as bacteroides and clostridia species, and acid-fast organisms such as Mycobacteria, for example M. tuberculosis; and/or Mycobacterium avium. Other examples include Escherichia, for example E. coli. intercellular microbes, for example Chlamydia and Rickettsiae.

Examples of infections that may be treated with the compounds of the present invention include central nervous system infections, external ear infections, infections of the middle ear, such as acute otitis media, infections of the cranial sinuses, eye infections, infections of the oral cavity, such as infections of the teeth, gums and mucosa, upper respiratory tract infections, lower respiratory tract infections, genitourinary infections, gastrointestinal infections, gynecological infections, septicemia, bone and joint infections, skin and skin structure infections, bacterial endocarditis, burns, antibacterial prophylaxis of surgery, and antibacterial prophylaxis in immunosuppressed patients, such as patients receiving cancer chemotherapy, or organ transplant patients. Specifically, infectious diseases that may be treated with the compounds of the present invention are gram-positive infections such as osteomyelitis, endocarditis and diabetic foot.

### Antibacterial Activity

The in vitro antibacterial activity of the compounds of the present invention may be assessed by following procedures recommended in (1) National Committee for Clinical Laboratory Standards (January 2003), Methods for dilution antimicrobial tests for bacteria that grow aerobically, Approved Standard (6^{th} ed), M7-A6, NCCLS, Wayne, Pa.; (2) National Committee for Clinical Laboratory Standards (March 2001), Methods for antimicrobial susceptibility testing of anaerobic bacteria, Approved Standard (5^{th} ed), M11-A4, NCCLS, Wayne, Pa.; (3) National Committee for Clinical Laboratory Standards (Jan. 2003), MIC testing supplemental tables, M100-S13 (for use with M7-A6), NCCLS, Wayne, Pa.; and (4) Murray P R, Baron E J, Jorgensen J H, et al. Manual of Clinical Microbiology (8^{th} ed) Washington, D.C.: American Society for Microbiology Press, 2003. The MIC value is the lowest concentration of drug which prevented macroscopically visible growth under the conditions of the test. Table 1 shows the in vitro testing results.

TABLE 1
Results of in vitro antibacterial activity MIC₉₀ (μg/mL)

| Example | S. aureus | S. pneumoniae | E. faecalis |
|---|---|---|---|
| No. | UC-76 SA-1 | SV1 SP-3 | MGH-2 EF 1-1 |
| 1 | 8 | 1 | 2 |
| 2 | 8 | 1 | 2 |
| 3 | 4 | 2 | 4 |
| 4 | 8 | 2 | 4 |
| 5 | 8 | 2 | 8 |
| 6 | 4 | 2 | 2 |
| 7 | 2 | 2 | 2 |
| 8 | 4 | 4 | 2 |
| 9 | 8 | 2 | 2 |
| 10 | 8 | 2 | 2 |
| 11 | 4 | 1 | 2 |
| 12 | 8 | 4 | 4 |
| 13 | 4 | 2 | 4 |
| 14 | 8 | 4 | 8 |
| 15 | 4 | 2 | 2 |
| 16 | 8 | 4 | 4 |
| 17 | 4 | 2 | 4 |
| 18 | 8 | 4 | 8 |
| 10 | 4 | 2 | 2 |
| 20 | 8 | 4 | 8 |
| 21 | 8 | 2 | 4 |
| 22 | 8 | 4 | 4 |
| 23 | 8 | 4 | 4 |
| 24 | 8 | 4 | 4 |
| 25 | 8 | 8 | 8 |
| 26 | 8 | 4 | 4 |
| 27 | 8 | 4 | 8 |
| 28 | 8 | 2 | 4 |
| 29 | 2 | 2 | 2 |

### Pharmaceutical Salts

The compound of formula I may be used in its native form or as a salt. In cases where forming a stable nontoxic acid or base salt is desired, administration of the compound as a pharmaceutically acceptable salt may be appropriate. Examples of pharmaceutically acceptable salts of the present invention include inorganic salts such as hydrochloride, hydrobromide, sulfate, nitrate, bicarbonate, carbonate salts, and organic salts such as tosylate, methanesulfonate, acetate, citrate, malonate, tartarate, succinate, benzoate, ascorbate, etoglutarate, and glycerophosphate.

Pharmaceutically acceptable salts may be obtained using standard procedures well known in the art, for example, reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

### Routes of Administration

The oxazolidinone antibacterial prodrugs of this invention have useful activity against a variety of organisms including, but not limiting to, Staphylococcus aureus, Staphylococcus epidermidis, Enterococcus faecium, Streptococcus pneumoniae, Streptococcus pyogenes, Enterococcus faecalis, Moraxella catarrhalis and H. influenzae. In therapeutic use for treating, or combating, bacterial infections in a mammal (i.e. human and animals) an oxazolidinone prodrug of the present invention or its pharmaceutical compositions can be administered orally, parenterally, topically, rectally, transmucosally, or intestinally.

Parenteral administrations include indirect injections to generate a systemic effect or direct injections to the afflicted area. Examples of parenteral administrations are subcutaneous, intravenous, intramuscular, intradermal, intrathecal, intraocular, intranasal, intravetricular injections or infusions techniques.

Topical administrations include the treatment of infectious areas or organs readily accessibly by local application, such as, for example, eyes, ears including external and middle ear infections, vaginal, open wound, skins including the surface skin and the underneath dermal structures, or other lower intestinal tract. It also includes transdermal delivery to generate a systemic effect.

The rectal administration includes the form of suppositories.

The transmucosal administration includes nasal aerosol or inhalation applications.

The preferred routes of administration are oral and parenteral.

### Composition/Formulation

Pharmaceutical compositions of the present invention may be manufactured by processes well known in the art, e.g., by means of conventional mixing, dissolving, granulation, dragee-making, levigating, emulsifying, encapsulating, entrapping, lyophilizing processes or spray drying.

Pharmaceutical compositions for use in accordance with the present invention may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

For oral administration, the compounds can be formulated by combining the active compounds with pharmaceutically acceptable carriers well known in the art. Such carriers enable the compounds of the invention to be formulated as tablets, pills, lozenges, dragees, capsules, liquids, solutions, emulsions, gels, syrups, slurries, suspensions and the like, for oral ingestion by a patient. A carrier can be at least one substance which may also function as a diluent, flavoring agent, solubilizer, lubricant, suspending agent, binder, tablet disintegrating agent, and encapsulating agent. Examples of such carriers or excipients include, but are not limited to, magnesium carbonate, magnesium stearate, talc, sugar, lactose, sucrose, pectin, dextrin, mannitol, sorbitol, starches, gelatin, cellulosic materials, low melting wax, cocoa butter or powder, polymers such as polyethylene glycols and other pharmaceutical acceptable materials.

Dragee cores are provided with suitable coatings. For this purpose, concentrated sugar solutions may be used which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions which can be used orally include push-fit capsules made of gelatin, as well as soft, sealed capsules made of gelatin and a plasticizer, such as glycerol or sorbitol. The push-fit capsules can contain the active ingredients in admixture with a filler such as lactose, a binder such as starch, and/or a lubricant such as talc or magnesium stearate and, optionally, stabilizers. In soft capsules, the active compounds may be dissolved or suspended in suitable liquids, such as fatty oils, liquid paraffin, liquid polyethylene glycols, cremophor, capmul, medium or long chain mono-, di- or triglycerides. Stabilizers may be added in these formulations, also.

Liquid form compositions include solutions, suspensions and emulsions. For example, there may be provided solutions of the compounds of this invention dissolved in water and water-propylene glycol and water-polyethylene glycol systems, optionally containing suitable conventional coloring agents, flavoring agents, stabilizers and thickening agents.

The compounds may also be formulated for parenteral administration, e.g., by injections, bolus injection or continuous infusion. Formulations for parenteral administration may be presented in unit dosage form, e.g., in ampoules or in multi-dose containers, with an added preservative. The compositions may take such forms as suspensions, solutions or emulsions in oily or aqueous vehicles, and may contain formulating materials such as suspending, stabilizing and/or dispersing agents.

For injection, the compounds of the invention may be formulated in aqueous solution, preferably in physiologically compatible buffers or physiological saline buffer. Suitable buffering agents include trisodium orthophosphate, sodium bicarbonate, sodium citrate, N-methylglucamine, L(+)-lysine and L(+)-arginine.

Parenteral administrations also include aqueous solutions of a water soluble form, such as, without limitation, a salt, of the active compound. Additionally, suspensions of the active compounds may be prepared in a lipophilic vehicle. Suitable lipophilic vehicles include fatty oils such as sesame oil, synthetic fatty acid esters such as ethyl oleate and triglycerides, or materials such as liposomes. Aqueous injection suspensions may contain substances which increase the viscosity of the suspension, such as sodium carboxymethyl cellulose, sorbitol, or dextran. Optionally, the suspension may also contain suitable stabilizers and/or agents that increase the solubility of the compounds to allow for the preparation of highly concentrated solutions.

Alternatively, the active ingredient may be in powder form for constitution with a suitable vehicle, e.g., sterile, pyrogen-free water, before use. For suppository administration, the compounds may also be formulated by mixing the agent with a suitable non-irritating excipient which is solid at room temperature but liquid at rectal temperature and therefore will melt in the rectum to release the drug. Such materials include cocoa butter, beeswax and other glycerides.

For administration by inhalation, compounds of the present invention can be conveniently delivered through an aerosol spray in the form of solution, dry powder, or suspensions. The aerosol may use a pressurized pack or a nebulizer and a suitable propellant. In the case of a pressurized aerosol, the dosage unit may be controlled by providing a valve to deliver a metered amount. Capsules and cartridges of, for example, gelatin for use in an inhaler may be formulated containing a power base such as lactose or starch.

For topical applications, the pharmaceutical composition may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion such as suspensions, emulsion, or cream containing the active components suspended or dissolved in one or more pharmaceutically acceptable carriers. Suitable carriers include, but are not limited to, mineral oil, sorbitan monostearate, polysorbate 60, cetyl esters wax, ceteary alcohol, 2-octyldodecanol, benzyl alcohol and water.

For ophthalmic and otitis uses, the pharmaceutical compositions may be formulated as micronized suspensions in isotonic, pH adjusted sterile saline, or preferably, as solutions in isotonic, pH adjusted sterile saline, either with or without a preservative such as a benzylalkonium chloride. Alternatively, for ophthalmic uses, the pharmaceutical compositions may be formulated in an ointment such as petrolatum.

In addition to the formulations described previously, the compounds may also be formulated as depot preparations. Such long acting formulations may be in the form of implants. A compound of this invention may be formulated for this route of administration with suitable polymers, hydrophobic materials, or as a sparing soluble derivative such as, without limitation, a sparingly soluble salt.

Additionally, the compounds may be delivered using a sustained-release system. Various sustained-release materials have been established and are well known by those skilled in the art. Sustained-release capsules may, depending on their chemical nature, release the compounds for 24 hours or for up to several days.

### Dosage

Pharmaceutical compositions suitable for use in the present invention include compositions wherein the active ingredients are contained in an amount sufficient to achieve the intended purpose, i.e., the treatment or prevent of infectious diseases. More specifically, a therapeutically effective amount means an amount of compound effective to prevent, alleviate or ameliorate symptoms of disease or prolong the survival of the subject being treated.

The quantity of active component, that is the compound of this invention, in the pharmaceutical composition and unit dosage form thereof may be varied or adjusted widely depending upon the manner of administration, the potency of the particular compound and the desired concentration. Determination of a therapeutically effective amount is well within the capability of those skilled in the art. Generally, the quantity of active component will range between 0.5% to 90% by weight of the composition.

Generally, a therapeutically effective amount of dosage of active component will be in the range of about 0.1 to about 400 mg/kg of body weight/day, more preferably about 1.0 to about 50 mg/kg of body weight/day. It is to be understood that the dosages may vary depending upon the requirements of each subject and the severity of the bacterial infection being treated. In average, the effective amount of active component is about 200 mg to 800 mg and preferable 600 mg per day.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

Also, it is to be understood that the initial dosage administered may be increased beyond the above upper level in order to rapidly achieve the desired plasma concentration. On the other hand, the initial dosage may be smaller than the optimum and the daily dosage may be progressively increased during the course of treatment depending on the particular situation. If desired, the daily dose may also be divided into multiple doses for administration, e.g., two to four times per day.

In cases of local administration or selective uptake, the effective local concentration of the drug may not be related to plasma concentration and other procedures know in the art may be used to determine the desired dosage amount.

### Oral Efficacy

### EXAMPLES

In the discussion above and in the examples below, the following abbreviations have the following meanings. If an abbreviation is not defined, it has its generally accepted meaning.
bm=broad multiplet
BOC=tert-butoxycarbonyl
bd=broad doublet
bs=broad singlet
CDI=1,1O-carbodiimidazole
d=doublet
dd=doublet of doublets
dq=doublet of quartets
dt=doublet of triplets
DMF=dimethylformamide
DMAP=dimethylaminopyridine
DMSO=dimethyl sulfoxide
eq.=equivalents
g=grams
h=hours
HPLC=high pressure liquid chromatography
HATU=N—[(dimethylamino)-1H-1,2,3-triazolo-[4,5-b]pyridin-1-yl-methylene]-N-methylmethanaminium hexafluorophosphate N-oxide
LG=leaving group
m=multiplet
M=molar
M %=mole percent
max=maximum
meq=milliequivalent
mg=milligram
ML=milliliter
mm=millimeter
mmol=millimol
q=quartet
s=singlet
t or tr=triplet
TBS=tributylsilyl
TFA=trifluoroacetic acid
THF=tetrahydrofuran
TLC=thin layer chromatography
p-TLC=preparative thin layer chromatography
μL=microliter
N=normality
MeOH=methanol
DCM=dichloromethane
HCl=hydrochloric acid
ACN=acetonitrile
MS=mass spectrometry
rt=room temperature
EtOAc=ethyl acetate
EtO=ethoxy
Ac=acetate
NMP=1-methyl-2-pyrrolidinone
μL=microliter
J=coupling constant
NMR=Nuclear magnetic resonance
MHz=megahertz
Hz=hertz
M/Z=mass to charge ratio
min=minutes
Boc=tert-butoxycarbonyl
CBZ=benzyloxycarbonyl
DCC=1,3-dicyclohexylcarbodiimide
PyBop=benzotriazole-1-yl-oxy-trispyrrolidinophosphonium hexafluorophosphate

### Preparation 1

### Preparation of 5-Amino-1-methyl-1,3-dihydro-indol-2-one

### Step 1: 1-Methyl-1,3-dihydro-indol-2-one

1-Methyl-1H-indole-2,3-dione (5.00 g, 31.0 mmol) is heated with neat hydrazine hydrate (30 ml) at 130° C. for 1.5 hours. The reaction mixture is cooled, diluted with ice water, and extracted with ethyl acetate. The extract is washed with brine, dried over sodium sulfate, and evaporated to give product as a yellowish brown solid (4.38 g, 96%); HPLC r.t. 3.69 min; MS for C₉H₉NO m/z 148.1 (M+H)⁺.

### Step 2: 1-Methyl-5-nitro-1,3-dihydro-indol-2-one

1-Methyl-1,3-dihydro-indol-2-one (Step 1, 2.10 g, 14.3 mmol) is added in portions to 70% nitric acid (10 mil) at −10° C. After the addition is complete, the reaction is allowed to warm to room temperature and then stirred for 5 hours. The mixture is diluted with ice water and the resulting precipitate filtered, washed with water, and dried under vacuum to give product as a brown solid (1.88 g, 70%); HPLC r.t. 3.97 min; MS for C₉H₈N₂O₃ m/z 193.9 (M+H)⁺.

### Step 3: 5-Amino-1-methyl-1,3-dihydro-indol-2-one

Iron powder (2.09 g, 37.46 mmol) is added in small portion to a mixture of 1-methyl-5-nitro-1,3-dihydro-indol-2-one (Step 2, 1.8 g, 9.36 mmol) and ammonium chloride (4.96 g, 93.6 mmol) in ethanol (100 ml) and water (50 ml) at 90° C. The reaction mixture is stirred vigorously and heated for 30 min, cooled to room temperature, and diluted with dichloromethane (200 ml). The mixture is filtered through celite, the organic layer separated and washed with water and brine, dried over sodium sulfate, and evaporated to give product as a dark brown solid (1.44 g, 94.7%); HPLC r.t. 1.06 min; MS for C₉H₁₀N₂O m/z 163.2 (M+H)⁺.

### Preparation 2

### Preparation of 7-fluoro-1-methyl-5-nitro-1,3-dihydro-indol-2-one

### Step 1: 7-Fluoro-1-methyl-1H-indole-2,3-dione

7-Fluoro-1H-indole-2,3-dione (prepared according to the method of Gassman as described in U.S. Pat. No. 4,188,325, 1.0 g, 6.05 mmol), iodomethane (1.13 ml, 18.2 mmol) and potassium carbonate (1.65 g, 12.1 mmol) in DMF (15 ml) are stirred at room temperature for 24 hours. The reaction mixture is diluted with ethyl acetate, washed with water and brine, dried (Na₂SO₄), and evaporated to give product as an orange solid (1.05 g, 97%); HPLC r.t. 3.79 min; MS for C₉H₆FNO₂ m/z 180.0 (M+H)⁺.

### Step 2: 7-Fluoro-1-methyl-1,3-dihydro-indol-2-one

7-Fluoro-1-methyl-1H-indole-2,3-dione (Step 1, 1.05 g, 5.86 mmol) is heated with neat hydrazine hydrate (10 ml) at 130° C. for 1 hour. The mixture is cooled, diluted with ice water and extracted with ethyl acetate. The extract is washed with brine, dried (Na₂SO₄), and evaporated to give product as a light yellow solid (0.895 g, 92%); HPLC r.t. 4.07 min; MS for C₉H₈FNO m/z 165.16 (M+H)⁺.

### Step 3: 7-Fluoro-1-methyl-5-nitro-1,3-dihydro-indol-2-one

7-Fluoro-1-methyl-1,3-dihydro-indol-2-one (Step 2, 0.89 g, 5.38 mmol) is added portionwise to 70% nitric acid (5 ml) at −10° C. After the addition is complete, the reaction is allowed to warm to room temperature and then stirred for 7 hours. The mixture is diluted with ice water and the resulting precipitate filtered, washed with water, and dried under vacuum to give product as a light brown solid (0.84 g, 74%); HPLC r.t. 4.32 min.

### Preparation 3

### Preparation of 5-amino-1-ethyl-1,3-dihydro-indol-2-one

### Step 1: 1-Ethyl-1H-indole-2,3-dione

1H-Indole-2,3-dione (5.00 g, 0.034 mol), iodoethane (5.44 ml, 0.068 mol) and potassium carbonate (9.28 g, 0.068 mol) in DMF (50 ml) are stirred at room temperature for 72 hours. The reaction mixture is diluted with ethyl acetate, washed with water and brine, dried (Na₂SO₄) and evaporated to give product as an orange solid (5.95 g, 100%); HPLC r.t. 3.96 min; MS for C₁₀H₉NO₂ m/z 176.1 (M+H)⁺.

### Step 2: 1-Ethyl-1,3-dihydro-indol-2-one

1-Ethyl-1H-indole-2,3-dione (Step 1, 5.60 g, 31.9 mmol) is heated with neat hydrazine hydrate (20 ml) at 130° C. for 1 hour. The reaction mixture is cooled, diluted with ice water, and extracted with ethyl acetate. The organic layer is washed with brine, dried (Na₂SO₄) and evaporated to give product as a yellowish orange solid (4.85 g, 94%); HPLC r.t. 4.12 min; MS for C₁₀H₁₁NO m/z 162.1 (M+H)⁺.

### Step 3: 1-Ethyl-5-nitro-1,3-dihydro-indol-2-one

1-Ethyl-1,3-dihydro-indol-2-one (Step 2, 4.00 g, 24.8 mmol) is added to a stirred solution of sodium nitrate (2.10 g, 24.8 mmol) in trifluoroacetic acid (100 ml). The reaction mixture is stirred at room temperature for 30 minutes and then poured on ice. The resulting precipitate was filtered, washed with water, and dried under vacuum to give product as a brown solid (3.7 g, 72%); HPLC r.t. 4.29 min; MS for C₁₀H₁₀N₂O₃ m/z 207.2 (M+H)⁺.

### Step 4: 5-Amino-1-ethyl-1,3-dihydro-indol-2-one

Iron powder (3.89 g, 69.8 mmol) is added portionwise to a mixture of 1-ethyl-5-nitro-1,3-dihydro-indol-2-one (Step 3, 3.60 g, 17.5 mmol) and ammonium chloride (9.24 g, 175 mmol) in ethanol (150 ml) and water (75 ml) at 90° C. The reaction mixture is stirred vigorously and heated for 30 minutes, cooled to room temperature and diluted with dichloromethane (300 ml). The mixture is filtered through celite, the organic layer separated and washed with water and brine, dried over sodium sulfate and evaporated to give product as a dark brown solid (2.74 g, 89%); HPLC r.t. 1.86 min; MS for C₁₀H₁₂N₂O m/z 177.1 (M+H)⁺.

### Preparation 4

### Preparation of 5-Amino-1-isopropyl-1,3-dihydro-indol-2-one

### Step 1: 1-Isopropyl-1H-indole-2,3-dione

1H-Indole-2,3-dione (5.0 g, 0.034 mol), iodopropane (6.83 ml, 0.068 mol) and potassium carbonate (9.28 g, 0.068 mol) in DMF (30 ml) are stirred at room temperature for 72 hours. The reaction mixture is diluted with ethyl acetate, washed with water and brine, dried (Na₂SO₄) and evaporated to give product as an orange solid (6.08 g, 94.5%); HPLC r.t. 4.38 min; MS for C₁₁H₁₁NO₂ m/z 190.1 (M+H)⁺.

### Step 2: 1-Isopropyl-1,3-dihydro-indol-2-one

1-Isopropyl-1H-indole-2,3-dione (Step 1, 3.00 g, 15.9 mmol) was heated with neat hydrazine hydrate (10 ml) at 130° C. for 1.5 hours. The reaction was cooled, diluted with ice water, and extracted with ethyl acetate. The organic layer is washed with brine, dried (Na₂SO₄), and evaporated to give product as a light brown solid (2.64 g, 95%); HPLC r.t. 4.54 min; MS for C₁₁H₁₃NO m/z 176.1 (M+H)⁺.

### Step 3: 1-Isopropyl-5-nitro-1,3-dihydro-indol-2-one

1-Isopropyl-1,3-dihydro-indol-2-one (Step 2, 2.50 g, 14.3 mmol) is added to a stirred solution of sodium nitrate (1.20 g, 14.26 mmol) in trifluoroacetic acid (50 ml) and stirred at room temperature for 5 h. The reaction was diluted with ice water and resulting precipitate filtered, washed with water, and dried under vacuum to give product as a brown solid (2.7 g, 86%); HPLC r.t. 4.71 min; MS for C₁₁H₁₂N₂O₃ M/Z 219.0 (M−H)⁻.

### Step 4: 5-Amino-1-isopropyl-1,3-dihydro-indol-2-one

Iron powder (2.63 g, 47.2 mmol) is added in small portion to a mixture of 1-isopropyl-5-nitro-1,3-dihydro-indol-2-one (Step 3, 2.60 g, 11.8 mmol) and ammonium chloride (6.27 g, 118 mmol) in ethanol (80 ml) and water (40 ml) at 90° C. The reaction mixture is stirred vigorously and heated for 45 min, then cooled to room temperature and diluted with dichloromethane (250 ml). The mixture is filtered through celite, the organic layer separated and washed with water and brine, dried (Na₂SO₄) and evaporated to give product as a dark brown gummy solid (2.2 g, 98.2%); HPLC r.t. 2.51 min; MS for C₁₁H₁₄N₂O m/z 191.1 (M+H)⁺.

### Preparation 5

### Preparation of 5-Amino-1-propyl-1,3-dihydro-indol-2-one

### Step 1: 5-Nitro-1-propyl-1,3-dihydro-indol-2-one

(2-Fluoro-5-nitrophenyl)acetic acid (Step 1, 5.00 g, 0.0251 mol) and n-propylamine (5 eq., 10.4 ml, 0.126 mol) are mixed in DMSO (25 ml) and stirred at 45° C. overnight. Excess n-propylamine is removed under vacuum and 2N hydrochloric acid (80 ml) added in one portion. The mixture is stirred for 20 minutes at room temperature and the resulting light yellow precipitate filtered, washed with water and dried under vacuum (5.9 g, 96%); HPLC r.t. 4.68 min MS for C11H12N2O3 mm/z 220.9 (M+H)⁺.

### Step 2: 5-Amino-1-propyl-1,3-dihydro-indol-2-one

Iron powder (3.30 g, 59 mmol) is added portionwise to 5-Nitro-1-propyl-1,3-dihydro-indol-2-one (Step 1, 3.25 g, 14.8 mmol) and ammonium chloride (7.8 g, 148 mmol) in ethanol (100 ml) and water (50 ml) at 90° C. The reaction is stirred vigorously and heated for 60 min, cooled to room temperature and diluted with dichloromethane (500 ml). The mixture is filtered through celite, the organic layer separated and washed with water and brine, dried (Na₂SO₄) and evaporated to give product as a dark brown solid (2.8 g, 99%); HPLC r.t. 2.62 min; MS for C11H14N2O m/z 191.1 (M+H)⁺.

### Preparation 6

### Preparation of (R)-3-(7-fluoro-2-oxo-1-propyl-2,3-dihydro-1H-indol-5-ylamino)-2-hydroxy-propionic acid methyl ester

### Step 1: 7-Fluoro-5-nitro-1-propyl-1,3-dihydro-indol-2-one

Crude (2,3-difluoro-5-nitrophenyl)acetic acid (Example 33, Step 1, 2.00 g, 9.2 mmol) and n-propylamine (6 eq., 4.54 ml, 0.0553 mol) are mixed in DMSO (10 ml) and stirred at 50° C. for 2 hours. 2N Hydrochloric acid (40 ml) is added in one portion and the mixture stirred at room temperature for 2 hours. The resulting light yellow precipitate is filtered, washed with water and dried under vacuum. The residue is purified by flash column chromatography (20% Ethylacetate/hexane) to give product as a yellow solid (0.93 g, 42% isolated yield, 85% assuming that starting material is 50% desired 5-NO₂ isomer); HPLC r.t. 5.40 min; MS for C11H11FN2O3 m/z 239.1 (M+H)⁺.

### Step 2: 5-Amino-7-fluoro-1-propyl-1,3-dihydro-indol-2-one

Iron powder (0.855 g, 15.3 mmol) is added in small portions to 7-Fluoro-5-nitro-1-propyl-1,3-dihydro-indol-2-one (Step 1, 0.910 g, 3.82 mmol) and ammonium chloride (2.02 g, 38.2 mmol) in ethanol (60 ml) and water (30 ml) at 90° C. The reaction mixture is stirred vigorously and heated for 60 min, cooled to room temperature, and diluted with dichloromethane (300 ml). The mixture is filtered through celite, the organic layer separated and washed with water and brine, dried over sodium sulfate and evaporated to give product as a dark brown solid (0.820 g, 99%); HPLC r.t. 3.03 min; MS for C11H13FN2O m/z 209.0 (M+H)⁺.

### Step 3: (R)-3-(7-Fluoro-2-oxo-1-propyl-2,3-dihydro-1H-indol-5-ylamino)-2-hydroxy-propionic acid methyl ester

5-Amino-7-fluoro-1-propyl-1,3-dihydro-indol-2-one (Step 2, 0.300 g, 1.44 mmol), methyl (2R)-glycidate (0.147 g, 1.44 mmol) and lithium trifluoromethanesulfonate (0.220 g, 1.44 mmol) in acetonitrile (5 ml) are heated at 90° C. for 8 hours. The reaction is diluted with ethyl acetate, washed with water and brine, dried (Na₂SO₄) and evaporated. The residue is purified by PTLC (5% methanol/dichloromethane) to give pure product as a yellow solid (0.250 g, 56%); HPLC r.t. 4.03 min; MS for C15H19FN2O₄ m/z 311.2 (M+H)⁺.

### Preparation 7

### Preparation of 5-Amino-1-cyclopropyl-1,3-dihydro-indol-2-one

### Step 1: (2-Fluoro-5-nitrophenyl)acetic acid

(2-Fluorophenyl)acetic acid (5 g, 0.0324 mol) is dissolved in concentrated sulfuric acid (20 ml) and the resulting solution cooled to −10° C. with vigorous stirring. A solution of nitric acid (2.08 ml, 69.3%, 0.0324 mol) and sulfuric acid (2 ml) is added dropwise at a rate such that the temperature remains below −5° C. The thickened slurry is stirred for 15 minutes and then poured on ice. The resulting white precipitate is filtered and dried under vacuum (6.5 g, 99%); ¹H NMR (300 mHz, DMSO-d₆) □8.35 (1H, dd), 8.26-8.18 (1H, m), 7.48 (1H, t), 3.80 (2H, d).

### Step 2: 1-Cyclopropyl-5-nitro-1,3-dihydro-indol-2-one

(2-Fluoro-5-nitrophenyl)acetic acid (Step 1, 1.00 g, 0.00502 mol) and cyclopropylamine (6 eq., 2.08 ml, 0.0301 mol) are mixed in DMSO (5 ml) and stirred at 45° C. overnight. Excess cyclopropylamine is removed under vacuum and 2N hydrochloric acid (20 ml) added in one portion. The mixture is stirred for 20 minutes at room temperature and the resulting light yellow precipitate filtered, washed with water and dried under vacuum (0.95 g, 87%).

### Step 3: 5-Amino-1-cyclopropyl-1,3-dihydro-indol-2-one

Iron powder (1.26 g, 22.9 mmol) is added portionwise to 1-cyclopropyl-5-nitro-1,3-dihydro-indol-2-one (Step 2, 1.25 g, 5.72 mmol) and ammonium chloride (3.01 g, 57.2 mmol) in ethanol (50 ml) and water (25 ml) at 90° C. The reaction is stirred vigorously and heated for 30 min, cooled to room temperature and diluted with dichloromethane (150 ml). The mixture is filtered through celite, the organic layer separated and washed with water and brine, dried (Na₂SO₄) and evaporated to give product as a dark brown solid (0.98 g, 91%); HPLC r.t. 2.21 min; MS for C₁₁H₁₂N₂O m/z 189.1 (M+H)⁺.

### Preparation 8

### Preparation of 5-mino-1-sec-butyl-1,3-dihydro-indol-2-one

### Step 1: 1-sec-Butyl-5-nitro-1,3-dihydro-indol-2-one

(2-Fluoro-5-nitrophenyl)acetic acid (Example 45, Step 1, 2.00 g, 10.0 mmol) and sec-butylamine (6 eq., 6.08 ml, 60.2 mmol) are mixed in dimethyl sulfoxide (10 ml) and stirred at 45° C. overnight. Excess sec-butylamine is removed under vacuum and 2N hydrochloric acid (40 ml) added in one portion. The mixture is stirred for 1.5 hours at 45° C. and then extracted with dichloromethane. The extract is washed with brine, dried (Na₂SO₄) and evaporated. The residue is purified by flash column chromatography to give product as a yellow solid (1.68 g, 71%); HPLC r.t. 5.05 min; MS for C12H14N2O3 m/z 235.3 (M+H)⁺.

Step 2: 5-Amino-1-sec-butyl-1,3-dihydro-indol-2-one

Iron powder (1.55 g, 28.0 mmol) is added portionwise to 1-sec-Butyl-5-nitro-1,3-dihydro-indol-2-one (Step 1, 1.64 g, 7.00 mmol) and ammonium chloride (3.70 g, 70 mmol) in ethanol (70 ml) and water (35 ml) at 90° C. The reaction is stirred vigorously and heated for 45 min, cooled to room temperature and diluted with dichloromethane (200 ml). The mixture is filtered through celite, the organic layer separated and washed with water and brine, dried (Na₂SO₄) and evaporated to give product as a dark brown solid (1.41 g, 99%); HPLC r.t. 2.80 min; MS for C12H16N2O m/z 205.1 (M+H)⁺.

### Preparation 9

### Preparation of 5-amino-1-cyclopropylmethyl-1,3-dihydro-indol-2-one

### Step 1: 1-Cyclopropylmethyl-5-nitro-1,3-dihydro-indol-2-one

(2-Fluoro-5-nitrophenyl)acetic acid (Example 45, Step 1, 1.15 g, 5.75 mmol) and (aminomethyl)cyclopropane (6 eq., 3.00 ml, 34.5 mmol) are mixed in dimethyl sulfoxide (6 ml) and stirred at 45° C. overnight. Excess (aminomethyl)cyclopropane is removed under vacuum and 2N hydrochloric acid (25 ml) added in one portion. The mixture is stirred for 1 hour at room temperature and the resulting precipitate filtered, washed with water, and dried to give product as a yellowish-brown solid (1.25 g, 94%); HPLC r.t. 4.87 min; MS for C12H12N2O3 m/z 233.1 (M+H)⁺.

### Step 2: 5-Amino-1-cyclopropylmethyl-1,3-dihydro-indol-2-one

Iron powder (1.15 g, 20.7 mmol) is added portionwise to 1-Cyclopropylmethyl-5-nitro-1,3-dihydro-indol-2-one (Step 1, 1.2 g, 5.16 mmol) and ammonium chloride (2.75 g, 51.6 mmol) in ethanol (50 ml) and water (25 ml) at 90° C. The reaction is stirred vigorously and heated for 30 min, cooled to room temperature and diluted with dichloromethane (250 ml). The mixture is filtered through celite, the organic layer separated and washed with water and brine, dried (Na₂SO₄) and evaporated to give product as a dark brown solid (1.09 g, 96%); HPLC r.t. 2.68 min; MS for C12H14N2O m/z 203.4 (M+H)⁺.

### Preparation 10

### Preparation of 10 5-Amino-1-(2-fluoro-1-methyl-ethyl)-1,3-dihydro-indol-2-one

### Step 1: Toluene-4-sulfonic acid 2-fluoro-1-methyl-ethyl ester

p-Toluenesulfonic anhydride (16.3 g, 49.9 mmol) is added portionwise to 1-fluoro-2-propanol (3.00 g, 38.4 mmol), triethylamine (16.1 ml, 115 mmol) and 4-(dimethylamino)pyridine (1.41 g, 11.5 mmol) in dichloromethane (30 ml) at 0° C., allowed to warm to room temperature, and then stirred for 2 hours. The mixture is diluted with dichloromethane, washed with citric acid and brine, dried (Na₂SO₄) and evaporated to give product as an oil (7.15 g, 80%).

### Step 2: 1-(2-Fluoro-1-methyl-ethyl)-1H-indole-2,3-dione

Isatin (2.70 g, 18.4 mmol), Toluene-4-sulfonic acid 2-fluoro-1-methyl-ethyl ester (Step 1, 6.40 g, 27.6 mmol) and potassium carbonate (7.61 g, 55.1 mmol) in dimethylformamide (20 ml) are stirred at 50° C. for 24 hours. The reaction is diluted with water and extracted with ethyl acetate. The extract is washed with brine, dried (Na₂SO₄) and evaporated. The residue is purified by flash column chromatography (30% ethyl acetate/hexane) to give product as an orange solid (2.40 g, 63%); HPLC r.t. 4.38 min; MS for C11H10FNO2 m/z 207.9 (M+H)⁺.

### Step 3: 1-(2-Fluoro-1-methyl-ethyl)-1,3-dihydro-indol-2-one

1-(2-Fluoro-1-methyl-ethyl)-1H-indole-2,3-dione (Step 2, 2.30 g, 11.1 mmol) is heated with neat hydrazine hydrate (20 ml) at 130° C. for 30 minutes. The reaction mixture is cooled, diluted with ice water, and extracted with ethyl acetate. The extract is washed with brine, dried over sodium sulfate, and evaporated to give product as a yellowish brown solid (1.78 g, 83%); HPLC r.t. 4.50 min.

### Step 4: 1-(2-Fluoro-1-methyl-ethyl)-5-nitro-1,3-dihydro-indol-2-one

1-(2-Fluoro-1-methyl-ethyl)-1,3-dihydro-indol-2-one (Step 3, 1.68 g, 8.69 mmol) is added in portions to sodium nitrate (0.737 g, 8.69 mmol) in trifluoroacetic acid (15 ml). After the addition is complete, the reaction is stirred at room temperature for 8 hours. The mixture is diluted with ice water and the resulting precipitate filtered, washed with water, and dried under vacuum. Final purification by flash column chromatography (30% ethyl acetate/hexane) gives product as a light yellow solid (0.760 g, 37%); HPLC r.t. 4.75 min; MS for C11H11FN2O3 m/z 239.1 (M+H)⁺.

### Step 5: 5-Amino-1-(2-fluoro-1-methyl-ethyl)-1,3-dihydro-indol-2-one

Iron powder (0.714 g, 12.8 mmol) is added in small portion to a mixture of 1-methyl-5-nitro-1,3-dihydro-indol-2-one (Step 4, 0.760 g, 3.19 mmol) and ammonium chloride (1.68 g, 31.9 mmol) in ethanol (50 ml) and water (25 ml) at 90° C. The reaction mixture is stirred vigorously and heated for 45 min, cooled to room temperature, and diluted with dichloromethane (250 ml). The mixture is filtered through celite, the organic layer separated and washed with water and brine, dried over sodium sulfate, and evaporated to give product as a dark brown solid (0.665 g, 99%); HPLC r.t. 2.50 min; MS for C11H13FN2O m/z 209.0 (M+H)⁺.

### Example 1

### Preparation of (S)—N-[3-(1-methyl-2-oxo-2,3-dihydro-1H-indol-5-yl)-2-oxo-oxazolidin-5-ylmethyl]-acetamide

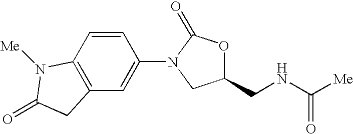

### Step 1: Preparation of (R)-[2-hydroxy-3-(1-Methyl-2-oxo-2,3-dihydro-1H-indol-5-ylamino)-propyl]-carbamic acid tert-butyl ester

5-Amino-1-methyl-1,3-dihydro-indol-2-one (Preparation 1, 1.20 g, 7.40 mmol), (S)-oxiranylmethyl-carbamic acid tert-butyl ester (1.28 g, 7.40 mmol) and lithium trifluoromethanesulfonate (1.14 g, 7.398 mmol) in acetonitrile (10 ml) are heated at 70° C. for 1 hour. The reaction mixture is diluted with ethyl acetate, washed with water and brine, dried (Na₂SO₄) and evaporated. The residue is purified by flash column chromatography (70% EtOAc/Hexane) to give the title compound as a light brown solid. HPLC r.t. 3.20 min; MS for C₁₇H₂₅N₃O₄ m/z 336.4 (M+H)⁺.

### Step 2: Preparation of (S)-[3-(1-methyl-2-oxo-2,3-dihydro-1H-indol-5-yl)-2-oxo-oxazolidin-5-ylmethyl]-carbamic acid tert-butyl ester

(R)-[2-Hydroxy-3-(1-methyl-2-oxo-2,3-dihydro-1H-indol-5-ylamino)-propyl]-carbamic acid tert.butyl ester (Step 1, 0.10 g, 0.298 mmol) and 1,1-carbonyldiimidazole (0.053 g, 0.327 mmol) in acetonitrile (3 ml) is stirred and heated at 60° C. for 2 hours. The reaction mixture is diluted with ethyl acetate, washed with water and brine, dried (Na₂SO₄) and evaporated. The residue was purified by PTLC (10% MeOH/DCM) to give the title compound as a light brown solid. HPLC r.t. 4.24 min; MS for C₁₈H₂₃N₃O₅ m/z 362.3 (M+H)⁺.

### Step 3: Preparation of (R)-(5-aminomethyl-2-oxo-oxazolidin-3-yl)-1-methyl-1,3-dihydro-indole-2-one

(S)-[3-(1-Methyl-2-oxo-2,3-dihydro-1H-indol-5-yl)-2-oxo-oxazolidin-5-ylmethyl]-carbamic acid tert-butyl ester (Step 2, 0.20 g, 0.553 mmol) is treated with 50% TFA/DCM (3 ml) for 30 minutes at room temperature. The reaction is evaporated to give the title compound as the TFA salt. HPLC r.t. 2.46 min; MS for C₁₃H₁₅N₃O₃ m/z 262.2 (M+H)⁺.

### Step 4: Preparation of (S)—N-[3-(1-methyl-2-oxo-2,3-dihydro-1H-indol-5-yl)-2-oxo-oxazolidin-5-ylmethyl]-acetamide

Acetic anhydride (0.016 ml, 0.166 mmol) is added to (R)-(5-aminomethyl-2-oxo-oxazolidin-3-yl)-1-methyl-1,3-dihydro-indole-2-one (Step 3, 0.041 g, 0.11 mmol) and diisopropyl ethylamine (0.057 ml, 0.33 mmol) in dichloromethane (3 ml)) at 0° C. The reaction is stirred at 0° C. for 30 minutes and then allowed to warm at room temperature. The mixture is diluted with dichloromethane, washed with water, citric acid and brine, dried (Na₂SO₄), and evaporated under vacuum to give the title compound as an off white solid. HPLC r.t. 3.013 min; ¹H NMR (300 MHz, CDCl₃) 7.58 (s, 1H), 7.27 (dd, J=2.1, 9.3 Hz, 1H), 6.79 (d, J=8.4 Hz, 1H), 6.01 (m, 1H), 4.73-4.82 (m, 1H), 4.05 (t, J=9 Hz, 1H), 3.79 (dd, J=6.9, 9.3 Hz, 1H), 3.57-3.76 (m, 2H), 3.54 (s, 2H), 3.21 (s, 3H), 2.03 (s, 3H); MS for C₁₅H₁₇N₃O₄ m/z 304 (M+H)⁺.

### Example 2

### Preparation of (S)—N-[3-(1-methyl-2-oxo-2,3-dihydro-1H-indol-5-yl)-2-oxo-oxazolidin-5-ylmethyl]-propionamide

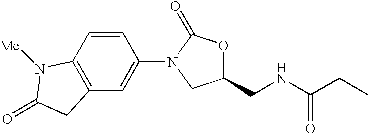

Propionic anhydride (0.024 ml, 0.186 mmol) is added to (R)-(5-aminomethyl-2-oxo-oxazolidin-3-yl)-1-methyl-1,3-dihydro-indole-2-one (Example 1, 0.035 g, 0.093 mmol) and diisopropylethylamine (0.097 ml, 0.559 mmol) in dichloromethane (3 ml) at 0° C. The reaction is allowed to warm to room temperature and stirred for 1 hour. The mixture is diluted with dichloromethane, washed with water, citric acid and brine, dried (Na₂SO₄), and evaporated. The residue is purified by PTLC (10% MeOH/DCM) to give the title compound as an off white solid. HPLC r.t. 3.200 min; ¹H NMR (300 MHz, CDCl₃) 7.57 (s, 1H), 7.27 (dd, J=2.1, 8.4 Hz, 1H), 6.79 (d, J=8.7 Hz, 1H), 6.07 (m, 1H), 4.73-4.81 (m, 1H), 4.04 (t, J=9 Hz, 1H), 3.81 (dd, J=6.3, 9 Hz, 1H), 3.64-3.71 (m, 2H), 3.53 (s, 2H), 3.20 (s, 3H), 2.25 (q, J=7.5 Hz, 2H), 1.13 (t, J=7.5 Hz, 3H); MS for C₁₆H₁₉N₃O₄ m/z 318 (M+H)⁺.

### Example 3

### Preparation of N-[3-(7-fluoro-1-methyl-2-oxo-2,3-dihydro-1H-indol-5-yl)-2-oxo-oxazolidin-5-ylmethyl]-acetamide

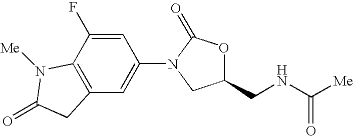

### Step 1: Preparation of (R)-[3-(7-fluoro-1-methyl-2-oxo-2,3-dihydro-1H-indol-5-ylamino)-2-hydroxy-propyl]-carbamic acid tert-butyl ester

5-Amino-7-fluoro-1-methyl-1,3-dihydro-indol-2-one (Preparation 2, 1.72 g, 9.54 mmol), (S)-oxiranylmethyl-carbamic acid tert-butyl ester (1.66 g, 9.54 mmol) and lithium trifluoromethanesulfonate (1.46 g, 9.54 mmol) in acetonitrile (15 ml) are heated at 90° C. for 2 hours. The reaction mixture is diluted with ethyl acetate, washed with water and brine, dried (Na₂SO₄) and evaporated. Final purification by flash chromatography (70% Ethyl acetate/hexane) gives the title compound as a yellow foamy solid. HPLC R.T. 3.75 min; MS for C₁₇H₂₄FN₃O₄ m/z 354 (M+H)⁺.

### Step 2: Preparation of (S)-[3-(7-fluoro-1-methyl-2-oxo-2,3-dihydro-1H-indol-5-yl)-2-oxo-oxazolidin-5-ylmethyl]-carbamic acid tert-butyl ester

Phosgene (20% solution in toluene, 2.70 ml, 0.0276 mol) is added to (R)-[3-(7-fluoro-1-methyl-2-oxo-2,3-dihydro-1H-indol-5-ylamino)-2-hydroxy-propyl]-carbamic acid tert-butyl ester (Step 5, 1.30 g, 0.00368 mol) and triethylamine (2.55 ml, 0.0184 mol) in dichloromethane (15 ml) at 0° C. and stirred for 30 minutes. The mixture is diluted with dichloromethane, washed with water and brine, dried (Na₂SO₄) and evaporated. The residue is purified by flash column chromatography (65 to 75% Ethyl acetate/hexane) to give the title compound as a light yellow solid. HPLC r.t. 4.68 min; MS for C₁₈H₂₂FN₃O₅ m/z 380 (M+H)⁺.

### Step 3: Preparation of (R)-5-(5-aminomethyl-2-oxo-oxazolidin-3-yl)-7-fluoro-1-methyl-1,3-dihydro-indol-2-one

(S)-[3-(7-Fluoro-1-methyl-2-oxo-2,3-dihydro-1H-indol-5-yl)-2-oxo-oxazolidin-5-ylmethyl]-carbamic acid tert-butyl ester (Step 6, 0.320 g, 0.843 mmol) is treated with 50% TFA/DCM (4 ml) for 15 minutes at room temperature. The reaction is evaporated and the title compound isolated as the TFA salt. HPLC r.t. 2.84 min; MS for C₁₃H₁₄FN₃O₃ m/z 280 (M+H)⁺.

### Step 4: Preparation of (S)—N-[3-(7-fluoro-1-methyl-2-oxo-2,3-dihydro-1H-indol-5-yl)-2-oxo-oxazolidin-5-ylmethyl]-acetamide

Acetic anhydride (0.115 ml, 1.22 mmol) is added to (R)-5-(5-aminomethyl-2-oxo-oxazolidin-3-yl)-7-fluoro-1-methyl-1,3-dihydro-indol-2-one (Step 7, 0.320 g, 0.813 mmol) and N,N-diisopropylethylamine (0.567 ml, 3.25 mmol) in dichloromethane (6 ml) at 0° C. The reaction is stirred at 0° C. for 30 minutes and then allowed to warm at room temperature. The mixture is diluted with dichloromethane, washed with water and brine, dried (Na₂SO₄) and evaporated. The residue is purified by PTLC (5% MeOH/DCM) to give the title compound as an off white solid. HPLC r.t. 3.39 min; ¹H NMR (300 MHz, CDCl₃) 7.27 (m, 1H), 7.23 (m, 1H), 5.96 (t, 1H), 5.98 (m, 1H), 4.74-4.80 (m, 1H), 4.02 (t, J=9 Hz, 1H), 3.76 (d, J=6.6, 9 Hz, 1H), 3.57-3.71 (m, 2H), 3.56 (s, 2H), 3.40 (d, J=2.7 Hz, 3H), 2.03 (s, 3H); MS for C₁₅H₁₆FN₃O₄ m/z 322.0 (M+H)⁺.

### Example 4

### Preparation of (S)—N-[3-(7-fluoro-1-methyl-2-oxo-2,3-dihydro-1H-indol-5-yl)-2-oxo-oxazolidin-5-ylmethyl]-propionamide

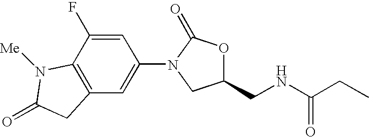

Propionic anhydride (0.202 ml, 1.57 mmol) is added dropwise to (R)-5-(5-aminomethyl-2-oxo-oxazolidin-3-yl)-7-fluoro-1-methyl-1,3-dihydro-indol-2-one (Example 3, 0.310 g, 0.788 mmol) and N,N-diisopropylethylamine (0.548 ml, 3.15 mmol) in dichloromethane (5 ml) at 0° C. The reaction is stirred at room temperature for 1 hour, then diluted with dichloromethane, washed with water and brine, dried (Na₂SO₄), and evaporated. The residue is purified by PTLC (5% methanol/dichloromethane) to give the title compound as a light yellow solid. HPLC r.t. 3.59 min; ¹H NMR (300 MHz, CDCl₃) 7.27 (m, 1H), 7.23 (m, 1H), 5.94 (t, 1H), 4.74-4.82 (m, 1H), 4.02 (t, J=8.7 Hz, 1H), 3.78 (dd, J=6.6, 9 Hz, 1H), 3.61-3.74 (m, 2H), 3.55 (s, 2H), 3.40 (d, J=2.4 Hz, 3H), 2.24 (q, J=7.5 Hz, 2H), 1.13 (t, J=7.5 Hz, 3H); MS for C₁₆H₁₈FN₃O₄ m/z 336.0 (M+H)⁺.

### Example 5

### Preparation of (S)—N-[3-(1-ethyl-2-oxo-2,3-dihydro-1H-indol-5-yl)-2-oxo-oxazolidin-5-ylmethyl]-acetamide

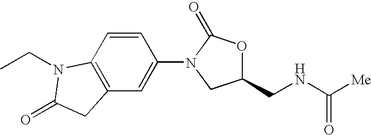

### Step 1: Preparation of (R)-[3-(1-ethyl-2-oxo-2,3-dihydro-1H-indol-5-ylamino)-2-hydroxy-propyl]-carbamic acid tert-butyl ester

5-Amino-1-ethyl-1,3-dihydro-indol-2-one (Preparation 3 0.91 g, 5.16 mmol), (S)-oxiranylmethyl-carbamic acid tert-butyl ester (0.893 g, 5.16 mmol) and lithium trifluoromethanesulfonate (0.798 g, 5.16 mmol) in acetonitrile (10 ml) are heated at 70° C. for 2 hours. The reaction mixture is diluted with ethyl acetate, washed with water and brine, dried (Na₂SO₄) and evaporated. Final purification by flash chromatography (30% Acetone/DCM) gives the title compound as a light brown foamy solid. HPLC R.T. 3.39 min; MS for C₁₈H₂₇N₃O₄ m/z 350.4 (M+H)⁺.

### Step 2: Preparation of (S)-[3-(1-ethyl-2-oxo-2,3-dihydro-1H-indol-5-yl)-2-oxo-oxazolidin-5-ylmethyl]-carbamic acid tert-butyl ester

Phosgene (20% solution in toluene, 0.140 ml, 1.43 mmol) is added to (R)-[3-(1-ethyl-2-oxo-2,3-dihydro-1H-indol-5-ylamino)-2-hydroxy-propyl]-carbamic acid tert-butyl ester (Step 1, 0.05 g, 0.143 mmol) and diisopropylethylamine (0.245 ml, 1.43 mmol) in dichloromethane (2 ml) at 0° C. The reaction is allowed to warm to room temperature and stirred for 2 hours. The mixture is diluted with dichloromethane, washed with water and brine, dried (Na₂SO₄) and evaporated. The residue is purified by PTLC (5% MeOH/DCM) to give the title compound as a light brown solid (0.04 g, 75%); HPLC r.t. 4.48 min; MS for C₁₉H₂₅N₃O₅ m/z 376.3 (M+H)⁺.

### Step 3: Preparation of (R)-(5-aminomethyl-2-oxo-oxazolidin-3-yl)-1-ethyl-1,3-dihydro-indole-2-one

(S)-[3-(1-Ethyl-2-oxo-2,3-dihydro-1H-indol-5-yl)-2-oxo-oxazolidin-5-ylmethyl]-carbamic acid tert-butyl ester (Step 2, 0.25 g, 0.665 mmol) is treated with 50% TFA/DCM (4 ml) for 30 minutes at room temperature. The reaction is evaporated and the title compound isolated as the TFA salt (0.257 g, 99%); HPLC r.t. 2.68 min; MS for C₁₄H₁₇N₃O₃ m/z 276.2 (M+H)⁺.

### Step 4: Preparation of (S)—N-[3-(1-ethyl-2-oxo-2,3-dihydro-1H-indol-5-yl)-2-oxo-oxazolidin-5-ylmethyl]-acetamide

Acetic anhydride (0.039 ml, 0.41 mmol) is added to (R)-(5-aminomethyl-2-oxo-oxazolidin-3-yl)-1-ethyl-1,3-dihydro-indole-2-one (Step 3, 0.080 g, 0.205 mmol) and pyridine (0.165 ml, 2.05 mmol) in dichloromethane (3 ml) at 0° C. The reaction is stirred at 0° C. for 30 minutes and then allowed to warm at room temperature. The mixture is diluted with dichloromethane, washed with water, citric acid and brine, dried (Na₂SO₄) and evaporated. The residue is purified by PTLC (5% MeOH/DCM) to give the title compound as an off white solid. HPLC r.t. 3.247 min; ¹H NMR (300 MHz, CDCl₃) 7.57 (s, 1H), 7.28 (dd, J=2.4, 8.7 Hz, 1H), 6.81 (d, J=8.4 Hz, 1H), 5.98 (m, 1H), 4.73-4.82 (m, 1H), 4.05 (t, J=8.7 Hz, 1H), 3.76 (q, J=7.5 Hz, 2H), 3.57-3.82 (m, 3H), 3.53 (s, 2H), 2.03 (s, 3H), 1.26 (t, J=7.5 Hz, 3H); MS for C₁₆H₁₉N₃O₄ m/z 318 (M+H)⁺.

### Example 6

### Preparation of (S)—N-[3-(1-ethyl-2-oxo-2,3-dihydro-1H-indol-5-yl)-2-oxo-oxazolidin-5-ylmethyl]-propionamide

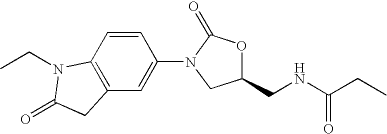

Propionic anhydride (0.042 ml, 0.334 mmol) is added dropwise to (R)-(5-aminomethyl-2-oxo-oxazolidin-3-yl)-1-ethyl-1,3-dihydro-indole-2-one (Preparation 3, 0.065 g, 0.167 mmol) and diisopropylethylamine (0.114 ml, 0.668 mmol) in dichloromethane (3 ml) at 0° C. The reaction is stirred at room temperature for 1 hour, then diluted with dichloromethane, washed with water, citric acid and brine, dried (Na₂SO₄), and evaporated to give the title compound as an off white solid.

HPLC r.t. 3.432 min; ¹H NMR (300 MHz, CDCl₃) 7.56 (s, 1H), 7.26 (dd, J=2.1, 8.4 Hz, 1H), 6.81 (d, J=8.1 Hz, 1H), 5.91 (m, 1H), 4.77 (m, 1H), 4.04 (t, J=9 Hz, 1H), 3.80 (dd, J=6, 9.3 Hz, 1H), 3.76 (q, J=7.2 Hz, 2H), 3.61-3.70 (m, 2H), 3.53 (s, 2H), 2.25 (q, J=7.5 Hz, 2H), 1.26 (t, J=7.5 Hz, 3H), 1.14 (t, J=7.5 Hz, 3H); MS for C₁₇H₂₁N₃O₄ m/z 332 (M+H)⁺.

### Example 7

### Preparation of (S)—N-[3-(1-ethyl-7-fluoro-2-oxo-2,3-dihydro-1H-indol-5-yl)-2-oxo-oxazolidin-5-ylmethyl]-acetamide

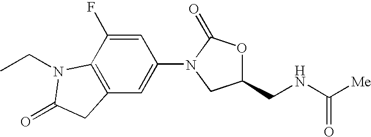

### Step 1: Preparation of 1-ethyl-7-fluoro-1H-indole-2,3-dione

7-Fluoro-1H-indole-2,3-dione (prepared according to the method of Gassman as described in U.S. Pat. No. 4,188,325 (4.00 g, 24.2 mmol), iodoethane (3.87 ml, 48.5 mmol) and potassium carbonate (6.61 g, 48.5 mmol) in DMF (20 ml) are stirred at room temperature for 20 hours. The reaction mixture is diluted with cold water and the resulting precipitate filtered and dried to give the title compound as an orange solid. HPLC r.t. 4.48 min; MS for C10H8FNO2 m/z 193.9 (M+H)⁺.

### Step 2: Preparation of 1-ethyl-7-fluoro-1,3-dihydro-indol-2-one

1-Ethyl-7-fluoro-1H-indole-2,3-dione (3.60 g, 18.6 mmol) is heated with neat hydrazine hydrate (15 ml) at 130° C. for 60 minutes. The mixture is cooled, diluted with ice water and extracted with ethyl acetate. The extract is washed with brine, dried (Na₂SO₄), and evaporated to give the title compound as an orange solid. HPLC r.t. 4.60 min; MS for C10H10FNO m/z 198.1 (M+H)⁺.

### Step 3: Preparation of 1-ethyl-7-fluoro-5-nitro-1,3-dihydro-indol-2-one

Preparation of 1-Ethyl-7-fluoro-1,3-dihydro-indol-2-one (3.45 g, 19.3 mmol) is added portionwise to a mixture of sodium nitrate (1.64 g, 19.3 mmol) and trifluoroacetic acid (60 ml) and stirred at room temperature for 5 hours. The reaction is diluted with ice water and the resulting precipitate filtered, washed with water, and dried under vacuum to give the title compound as a brownish-yellow solid (3.87 g, 90%); HPLC r.t. 4.88 min; MS for C10H9FN2O3 m/z 223.0 (M−H)⁻.

### Step 4: Preparation of 5-amino-1-ethyl-7-fluoro-1,3-dihydro-indol-2-one

Iron powder (3.84 g, 68.7 mmol) is added in small portions to 1-Ethyl-7-fluoro-5-nitro-1,3-dihydro-indol-2-one (Step 3, 3.85 g, 17.2 mmol) and ammonium chloride (8.94 g, 171 mmol) in ethanol (150 ml) and water (75 ml) at 90° C. The reaction mixture is stirred vigorously and heated for 30 min, cooled to room temperature, and diluted with dichloromethane (600 ml). The mixture is filtered through celite, the organic layer separated and washed with water and brine, dried over sodium sulfate and evaporated to give the title compound as a yellow solid. HPLC r.t. 2.60 min; MS for C10H11FN2O m/z 195.0 (M+H)⁺.

### Step 5: Preparation of (R)-[3-(1-ethyl-7-fluoro-2-oxo-2,3-dihydro-1H-indol-5-ylamino)-2-hydroxy-propyl]-carbamic acid tert-butyl ester

5-Amino-1-ethyl-7-fluoro-1,3-dihydro-indol-2-one (Step 4, 2.25 g, 11.6 mmol), (S)-oxiranylmethyl-carbamic acid tert-butyl ester (2.00 g, 11.6 mmol) and lithium trifluoromethanesulfonate (1.80 g, 11.6 mmol) in acetonitrile (12 ml) are heated at 90° C. for 20 hours. The reaction mixture is diluted with ethyl acetate, washed with water and brine, dried (Na₂SO₄) and evaporated. Final purification by flash chromatography (70% Ethyl acetate/hexane) gives the title compound as a yellow-brown foamy solid. HPLC r.t. 3.95 min; MS for C18H26FN3O4 m/z 368.3 (M+H)⁺.

### Step 6: Preparation of (S)-[3-(1-ethyl-7-fluoro-2-oxo-2,3-dihydro-1H-indol-5-yl)-2-oxo-oxazolidin-5-ylmethyl]-carbamic acid tert-butyl ester

Phosgene (20% solution in toluene, 3.58 ml, 36.7 mmol) is added to (R)-[3-(1-Ethyl-7-fluoro-2-oxo-2,3-dihydro-1H-indol-5-ylamino)-2-hydroxy-propyl]-carbamic acid tert-butyl ester (Step 5, 1.80 g, 4.89 mmol) and triethylamine (3.38 ml, 24.5 mmol) in dichloromethane (20 ml) at 0° C. and stirred for 30 minutes. The mixture is diluted with dichloromethane, washed with water and brine, dried (Na₂SO₄) and evaporated to give the title compound as light brown solid. HPLC r.t. 4.92 min; MS for C19H24FN3O5 m/z 394.1 (M+H)⁺.

### Step 7: Preparation of (R)-5-(5-aminomethyl-2-oxo-oxazolidin-3-yl)-1-ethyl-7-fluoro-1,3-dihydro-indol-2-one

(S)-[3-(1-Ethyl-7-fluoro-2-oxo-2,3-dihydro-1H-indol-5-yl)-2-oxo-oxazolidin-5-ylmethyl]-carbamic acid tert-butyl ester (Step 6, 0.325 g, 0.826 mmol) is treated with 50% TFA/DCM (4 ml) for 15 minutes at room temperature. The reaction is evaporated and the title compound isolated as the TFA salt. HPLC r.t. 3.13 min; MS for C14H16FN3O3 m/z 294.0 (M+H)⁺.

### Step 8: Preparation of (s) —N-[3-(1-ethyl-7-fluoro-2-oxo-2,3-dihydro-1H-indol-5-yl)-2-oxo-oxazolidin-5-ylmethyl]-acetamide

Acetic anhydride (0.116 ml, 1.23 mmol) is added to (R)-5-(5-aminomethyl-2-oxo-oxazolidin-3-yl)-1-ethyl-7-fluoro-1,3-dihydro-indol-2-one (Step 7, 0.335 g, 0.822 mmol) and N,N-diisopropylethylamine (0.572 ml, 3.28 mmol) in dichloromethane (5 ml) at 0° C. The reaction is stirred at 0° C. for 30 minutes and then allowed to warm at room temperature. The mixture is diluted with dichloromethane, washed with water and brine, dried (Na₂SO₄) and evaporated. The residue is purified by PTLC (5% MeOH/DCM) to give the title compound as an off white solid. HPLC r.t. 3.67 min; ¹H NMR (400 MHz, CDCl₃) 7.27 (m, 1H), 7.23 (dd, J=2, 13.2 Hz, 1H), 6.04 (t, 1H), 4.76-4.82 (m, 1H), 4.02 (t, J=9.2 Hz, 1H), 3.89 (q, J=7.2 Hz, 2H), 3.77 (dd, J=6.8, 9.2 Hz, 1H), 3.59-3.74 (m, 2H), 3.56 (s, 2H), 2.03 (s, 3H), 1.28 (t, J=7.2 Hz, 3H); MS for C₁₆H₁₈FN₃O₄ m/z 336.0 (M+H)⁺.

### Example 8

### Preparation of (S)—N-[3-(1-ethyl-7-fluoro-2-oxo-2,3-dihydro-1H-indol-5-yl)-2-oxo-oxazolidin-5-ylmethyl]-propionamide

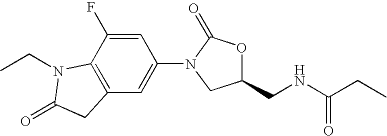

Propionic anhydride (0.193 ml, 1.52 mmol) is added dropwise to (R)-5-(5-Aminomethyl-2-oxo-oxazolidin-3-yl)-1-ethyl-7-fluoro-1,3-dihydro-indol-2-one (Example 7, 0.310 g, 0.762 mmol) and diisopropylethylamine (0.532 ml, 3.05 mmol) in dichloromethane (5 ml) at 0° C. The reaction is stirred at room temperature for 1 hour, then diluted with dichloromethane, washed with water and brine, dried (Na₂SO₄), and evaporated. The residue is purified by PTLC (5% MeOH/DCM) to give the title compound as a yellow solid. HPLC r.t. 3.88 min; ¹H NMR (400 MHz, CDCl₃) 7.27 (m, 1H), 7.23 (dd, J=2.1, 13.2 Hz, 1H), 5.96 (t, J=6 Hz, 1H), 4.75-4.81 (m, 1H), 4.02 (t, J=8.8 Hz, 1H), 3.89 (q, J=6.8 Hz, 2H), 3.79 (dd, J=6.8, 9.2 Hz, 1H), 3.61-3.74 (m, 2H), 3.55 (s, 2H), 2.25 (q, J=8 Hz, 2H), 1.28 (t, J=7.2 Hz, 3H), 1.14 (t, J=7.6 Hz, 3H); MS for C₁₇H₂₀FN₃O₄ m/z 350.0 (M+H)⁺.

### Example 9

### Preparation of (S)—N-{3-[1-(2-fluoro-ethyl)-2-oxo-2,3-dihydro-1H-indol-5-yl]-2-oxo-oxazolidin-5-ylmethyl}-acetamide

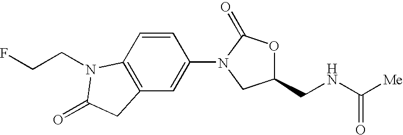

### Step 1: Preparation of (R)-{3-[1-(2-fluoro-ethyl)-2-oxo-2,3-dihydro-1H-indol-5-ylamino]-2-hydroxy-propyl}-carbamic acid tert-butyl ester

5-Amino-1-(2-fluoro-ethyl)-1,3-dihydro-indol-2-one (Example 18, Step 4, 1.00 g, 5.15 mmol), (S)-oxiranylmethyl-carbamic acid tert-butyl ester (0.894 g, 5.15 mmol) and lithium trifluoromethanesulfonate (0.793 g, 5.15 mmol) in acetonitrile (10 ml) are heated at 90° C. for 1 hour. The reaction mixture is diluted with ethyl acetate, washed with water and brine, dried (Na₂SO₄) and evaporated. Final purification by flash chromatography (70% ethyl acetate/hexane) gives pure product as a light yellow-brown solid. HPLC r.t. 3.28 min; MS for C18H26FN3O4 m/z 368.3 (M+H)⁺.

### Step 2: Preparation of (S)-{3-[1-(2-fluoro-ethyl)-2-oxo-2,3-dihydro-1H-indol-5-yl]-2-oxo-oxazolidin-5-ylmethyl}-carbamic acid tert-butyl ester

Phosgene (20% solution in toluene, 0.532 ml, 5.44 mmol) is added to (R)-{3-[1-(2-fluoro-ethyl)-2-oxo-2,3-dihydro-1H-indol-5-ylamino]-2-hydroxy-propyl}-carbamic acid tert-butyl ester (0.20 g, 0.544 mmol) and triethylamine (0.378 ml, 2.72 mmol) in dichloromethane (5 ml) at 0° C. and stirred for 30 minutes. The mixture is diluted with dichloromethane, washed with water and brine, dried (Na₂SO₄) and evaporated to give the title compound as a light brown solid. HPLC r.t. 4.37 min; MS for C19H24FN3O5 m/z 394.1 (M+H)⁺.

### Step 3: Preparation of (R)-5-(5-aminomethyl-2-oxo-oxazolidin-3-yl)-1-(2-fluoro-ethyl)-1,3-dihydro-indol-2-one

(S)-{3-[1-(2-Fluoro-ethyl)-2-oxo-2,3-dihydro-1H-indol-5-yl]-2-oxo-oxazolidin-5-ylmethyl}-carbamic acid tert-butyl ester (0.110 g, 0.280 mmol) is treated with 50% TFA/DCM (3 ml) for 15 minutes at room temperature. The reaction is evaporated and the product isolated as the TFA salt. HPLC r.t. 2.61 min; MS for C14H16FN3O3 m/z 294.0 (M+H)⁺.

### Step 4: Preparation of (S)—N-[3-(1-ethyl-7-fluoro-2-oxo-2,3-dihydro-1H-indol-5-yl)-2-oxo-oxazolidin-5-ylmethyl]-acetamide

Acetic anhydride (0.041 ml, 0.419 mmol) is added to (R)-5-(5-Aminomethyl-2-oxo-oxazolidin-3-yl)-1-(2-fluoro-ethyl)-1,3-dihydro-indol-2-one (Step 3, 0.114 g, 0.280 mmol) and N,N-diisopropylethylamine (0.194 ml, 1.12 mmol) in dichloromethane (5 ml) at 0° C. The reaction is stirred at 0° C. for 30 minutes and then allowed to warm at room temperature. The mixture is diluted with dichloromethane, washed with water and brine, dried (Na₂SO₄) and evaporated. The residue is purified by PTLC (10% MeOH/DCM) to give pure product as an off white solid. HPLC r.t. 3.15 min; ¹H NMR (300 MHz, CDCl₃) 7.61 (m, 1H), 7.23 (m, 1H), 6.91 (m, 1H), 5.96 (t, 1H), 4.75 (m, 1H), 4.74 (t, J=4.8 Hz, 1H), 4.59 (t, J=4.5 Hz, 1H), 4.06 (t, J=4.8 Hz, 1H), 4.04 (t, J=9 Hz, 1H), 3.97 (t, J=4.8 Hz, 1H), 3.78 (dd, J=6.9, 9 Hz, 1H), 3.61-3.75 (m, 2H), 3.58 (s, 2H), 2.03 (s, 3H); MS for C₁₆H₁₈FN₃O₄ m/z 336.0 (M+H)⁺.

### Example 10

### Preparation of (S)—N-{3-[1-(2-fluoro-ethyl)-2-oxo-2,3-dihydro-1H-indol-5-yl]-2-oxo-oxazolidin-5-ylmethyl}-propionamide

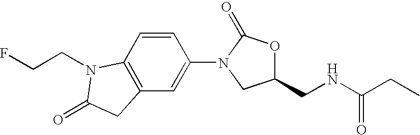

Propionic anhydride (0.072 ml, 0.559 mmol) is added dropwise to (R)-5-(5-aminomethyl-2-oxo-oxazolidin-3-yl)-1-(2-fluoro-ethyl)-1,3-dihydro-indol-2-one (Example 9, 0.114 g, 0.279 mmol) and diisopropylethylamine (0.194 ml, 1.12 mmol) in dichloromethane (4 ml) at 0° C. The reaction is stirred at room temperature for 1 hour, then diluted with dichloromethane, washed with water and brine, dried (Na₂SO₄), and evaporated. The residue is purified by PTLC (10% MeOH/DCM) to give the title compound as a yellow solid. HPLC r.t. 3.31 min; ¹H NMR (300 MHz, CDCl₃) 7.61 (m, 1H), 7.22-7.26 (m, 1H), 6.91 (d, J=8.4 Hz, 1H), 5.93 (t, 1H), 4.78 (m, 1H), 4.74 (t, J=4.8 Hz, 1H), 4.59 (t, J=4.5 Hz, 1H), 4.06 (t, J=4.8 Hz, 1H), 4.04 (t, J=9 Hz, 1H), 3.97 (t, J=5.1 Hz, 1H), 3.80 (dd, J=6.6, 9 Hz, 1H), 3.61-3.76 (m, 2H), 3.58 (s, 2H), 2.25 (q, J=7.5 Hz, 2H), 1.14 (t, J=7.5 Hz, 3H); MS for C₁₇H₂₀FN₃O₄ m/z 350.0 (M+H)⁺.

### Example 11

### Preparation of (S)—N-{3-[7-fluoro-1-(2-fluoro-ethyl)-2-oxo-2,3-dihydro-1H-indol-5-yl]-2-oxo-oxazolidin-5-ylmethyl}-acetamide

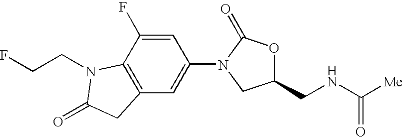

### Step 1: Preparation of 7-fluoro-1-(2-fluoro-ethyl)-1H-indole-2,3-dione

7-Fluoro-1H-indole-2,3-dione (prepared according to the method of Gassman as described in U.S. Pat. No. 4,188,325 (2.00 g, 12.1 mmol), 1-iodo-2-fluoroethane (4.21 g, 24.2 mmol) and potassium carbonate (3.30 g, 24.2 mmol) in DMF (20 ml) are stirred at room temperature for 20 hours. The reaction mixture is diluted with cold water and the resulting precipitate filtered and dried to give the title compound as an orange solid. HPLC r.t. 4.18 min; MS for C10H7F2NO2 m/z 212.1 (M+H)⁺.

### Step 2: Preparation of 7-fluoro-1-(2-fluoro-ethyl)-1,3-dihydro-indol-2-one

7-Fluoro-1-(2-fluoro-ethyl)-1H-indole-2,3-dione (1.04 g, 4.92 mmol) is heated with neat hydrazine hydrate (10 ml) at 130° C. for 20 minutes. The mixture is cooled, diluted with ice water and extracted with ethyl acetate. The extract is washed with brine, dried (Na₂SO₄), and evaporated to give the title compound as a brown solid. HPLC r.t. 4.36 min; MS for C10H9F2NO m/z 198.1 (M+H)⁺.

### Step 3: Preparation of 7-fluoro-1-(2-fluoro-ethyl)-5-nitro-1,3-dihydro-indol-2-one

7-Fluoro-1-(2-fluoro-ethyl)-1,3-dihydro-indol-2-one (0.880 g, 0.0119 mol) is added portionwise to 70% nitric acid (8 ml) at −10° C. After the addition is complete, the reaction is allowed to warm to room temperature and then stirred for 24 hours. The mixture is diluted with ice water and the resulting precipitate filtered, washed with water, and dried under vacuum to give the title compound as a light brown solid. HPLC r.t. 4.65 min; MS for C10H8F2N2O3 m/z 243.3 (M+H)⁺.

### Step 4: Preparation of 5-Amino-7-fluoro-1-(2-fluoro-ethyl)-1,3-dihydro-indol-2-one

Iron powder (0.416 g, 7.43 mmol) is added in small portions to 7-Fluoro-1-(2-fluoro-ethyl)-5-nitro-1,3-dihydro-indol-2-one (0.45 g, 1.86 mmol) and ammonium chloride (0.987 g, 18.6 mmol) in ethanol (40 ml) and water (20 ml) at 90° C. The reaction mixture is stirred vigorously and heated for 30 min, cooled to room temperature, and diluted with dichloromethane (100 ml). The mixture is filtered through celite, the organic layer separated and washed with water and brine, dried over sodium sulfate and evaporated to give the title compound as a dark brown solid. HPLC r.t. 2.34 min; MS for C10H10F2N2O m/z 213.2 (M+H)⁺.

### Step 5: Preparation of (R)-{3-[7-fluoro-1-(2-fluoro-ethyl)-2-oxo-2,3-dihydro-1H-indol-5-ylamino]-2-hydroxy-propyl}-carbamic acid tert-butyl ester

5-Amino-7-fluoro-1-(2-fluoro-ethyl)-1,3-dihydro-indol-2-one (0.39 g, 1.84 mmol), (S)-oxiranylmethyl-carbamic acid tert-butyl ester (0.319 g, 1.84 mmol) and lithium trifluoromethanesulfonate (0.283 g, 1.84 mmol) in acetonitrile (4 ml) are heated at 90° C. for 6 hours. The reaction mixture is diluted with ethyl acetate, washed with water and brine, dried (Na₂SO₄) and evaporated. Final purification by flash chromatography (70% Ethyl acetate/hexane) gives the title compound as a yellow-brown foamy solid. HPLC r.t. 3.92 min; MS for C18H25F2N3O4 m/z 386.1 (M+H)⁺.

### Step 6: Preparation of (S)-{3-[7-fluoro-1-(2-fluoro-ethyl)-2-oxo-2,3-dihydro-1H-indol-5-yl]-2-oxo-oxazolidin-5-ylmethyl}-carbamic acid tert-butyl ester

Phosgene (20% solution in toluene, 0.414 ml, 4.24 mmol) is added to (R)-{3-[7-Fluoro-1-(2-fluoro-ethyl)-2-oxo-2,3-dihydro-1H-indol-5-ylamino]-2-hydroxy-propyl}-carbamic a cid tert-butyl ester (Step 5, 0.150 g, 0.424 mmol) and triethylamine (0.295 ml, 2.12 mmol) in dichloromethane (3 ml) at 0° C. and stirred for 30 minutes. The mixture is diluted with dichloromethane, washed with water and brine, dried (Na₂SO₄) and evaporated to give the title compound as a light brown solid. HPLC r.t. 4.70 min; MS for C19H23F2N3O5 m/z 380.3 (M+H)⁺.

### Step 7: Preparation of (R)-5-(5-aminomethyl-2-oxo-oxazolidin-3-yl)-7-fluoro-1-(2-fluoro-ethyl)-1,3-dihydro-indol-2-one

(S)-{3-[7-Fluoro-1-(2-fluoro-ethyl)-2-oxo-2,3-dihydro-1H-indol-5-yl]-2-oxo-oxazolidin-5-ylmethyl}-carbamic acid tert-butyl ester (Step 6, 0.15 g, 0.395 mmol) is treated with 50% TFA/DCM (3 ml) for 15 minutes at room temperature. The reaction is evaporated and the title compound isolated as the TFA salt. HPLC r.t. 2.86 min; MS for C14H15F2N3O3 m/z 280.0 (M+H)⁺.

### Step 8: Preparation of (S)—N-{3-[7-fluoro-1-(2-fluoro-ethyl)-2-oxo-2,3-dihydro-1H-indol-5-yl]-2-oxo-oxazolidin-5-ylmethyl}-acetamide

Acetic anhydride (0.083 ml, 0.856 mmol) is added to (R)-5-(5-Aminomethyl-2-oxo-oxazolidin-3-yl)-7-fluoro-1-(2-fluoro-ethyl)-1,3-dihydro-indol-2-one (Step 7, 0.243 g, 0.571 mmol) and N,N-diisopropylethylamine (0.395 ml, 2.28 mmol) in dichloromethane (5 ml) at 0° C. The reaction is stirred at 0° C. for 30 minutes and then allowed to warm at room temperature. The mixture is diluted with dichloromethane, washed with water and brine, dried (Na₂SO₄) and evaporated. The residue is purified by PTLC (10% MeOH/DCM) to give the title compound as an off white solid. HPLC r.t. 3.50 min; ¹H NMR (300 MHz, CDCl₃) 7.30 (m, 1H), 7.25 (m, 1H), 6.02 (t, 1H), 4.78 (m, 1H), 4.73 (t, J=4.5 Hz, 1H), 4.57 (t, J=4.8 Hz, 1H), 4.22 (t, J=4.8 Hz, 1H), 4.14 (t, J=4.8 Hz, 1H), 4.02 (t, J=9.3 Hz, 1H), 3.77 (dd, J=6.6, 9 Hz, 1H), 3.63-3.72 (m, 2H), 3.61 (s, 2H), 2.01 (s, 3H); MS for C₁₆H₁₇F₂N₃O₄ m/z 354.2 (M+H)⁺.

### Example 12

### Preparation of (S)—N-{3-[7-fluoro-1-(2-fluoro-ethyl)-2-oxo-2,3-dihydro-1H-indol-5-yl]-2-oxo-oxazolidin-5-ylmethyl}-propionamide

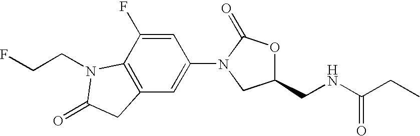

Propionic anhydride (0.056 ml, 0.437 mmol) is added dropwise to (R)-5-(5-Aminomethyl-2-oxo-oxazolidin-3-yl)-7-fluoro-1-(2-fluoro-ethyl)-1,3-dihydro-indol-2-one (Example 23, Step 7, 0.093 g, 0.218 mmol) and diisopropylethylamine (0.151 ml, 0.874 mmol) in dichloromethane (2 ml) at 0° C. The reaction is stirred at room temperature for 1 hour, then diluted with dichloromethane, washed with water and brine, dried (Na₂SO₄), and evaporated to give the title compound as an off white solid. HPLC r.t. 3.72 min; ¹H NMR (300 MHz, CDCl₃) 7.30 (m, 1H), 7.25 (dd, 1H), 5.94 (t, 1H), 4.79 (m, 1H), 4.73 (t, J=4.5 Hz, 1H), 4.57 (t, J=5.1 Hz, 1H), 4.22 (t, J=3.9 Hz, 1H), 4.14 (t, J=4.2 Hz, 1H), 4.02 (t, J=9 Hz, 1H), 3.79 (dd, J=6.3, 9 Hz, 1H), 3.65-3.74 (m, 2H), 3.61 (s, 2H), 2.24 (q, J=7.5 Hz, 2H), 1.14 (t, J=7.5 Hz, 3H); MS for C₁₇H₁₉F₂N₃O₄ m/z 368.3 (M+H)⁺.

### Example 13

### Preparation of (S)—N-[3-(1-isopropyl-2-oxo-2,3-dihydro-1H-indol-5-yl)-2-oxo-oxazolidin-5-ylmethyl]-acetamide

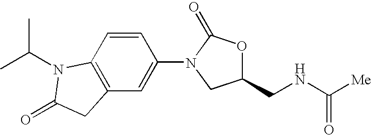

### Step 1: Preparation of (R)-[2-hydroxy-3-(1-isopropyl-2-oxo-2,3-dihydro-1H-indol-5-ylamino)-propyl]-carbamic acid tert-butyl ester

5-Amino-1-isopropyl-1,3-dihydro-indol-2-one (Preparation 4, 0.76 g, 3.99 mmol), (S)-oxiranylmethyl-carbamic acid tert-butyl ester (0.69 g, 3.99 mmol) and lithium trifluoromethanesulfonate (0.617 g, 3.99 mmol) in acetonitrile (10 ml) is heated at 70° C. for 2 hours. The reaction is diluted with ethyl acetate, washed with water and brine, dried (Na₂SO₄) and evaporated. Final purification by flash chromatography (20% Acetone/DCM) gives the title compound as a light brown foamy solid. HPLC r.t. 3.61 min; MS for C₁₉H₂₉N₃O₄ m/z 364.0 (M+H)⁺.

### Step 2: Preparation of (S)-[3-(1-isopropyl-2-oxo-2,3-dihydro-1H-indol-5-yl)-2-oxo-oxazolidin-5-ylmethyl]-carbamic acid tert-butyl ester

Phosgene (20% solution in toluene, 0.602 ml, 6.18 mmol) is added to (5R)-[2-hydroxy-3-(1-isopropyl-2-oxo-2,3-dihydro-1H-indol-5-ylamino)-propyl]-carbamic acid tert-butyl ester (0.15 g, 0.412 mmol) and triethylamine (0.28 ml, 2.06 mmol) in dichloromethane (5 ml) at 0° C. The reaction is allowed to warm to room temperature and stirred for 2 h. The mixture is diluted with dichloromethane, washed with water and brine, dried (Na₂SO₄) and evaporated. Final purification by PTLC (5% MeOH/DCM) gives the title compound as a light brown solid. HPLC r.t. 4.78 min; MS for C₂₀H₂₇N₃O₅ m/z 390.3 (M+H)⁺.

### Step 3: Preparation of (R)-(5-aminomethyl-2-oxo-oxazolidin-3-yl)-1-isopropyl-1,3-dihydro-indole-2-one

(5R)-[3-(1-Isopropyl-2-oxo-2,3-dihydro-1H-indol-5-yl)-2-oxo-oxazolidin-5-ylmethyl]-carbamic acid tert-butyl ester (0.25 g, 0.642 mmol) is treated with 50% TFA/DCM (4 ml) for 30 minutes at room temperature. The reaction is evaporated to give the title compound as a TFA salt. HPLC r.t. 3.06 min; MS for C₁₅H₁₉N₃O₃ m/z 290.2 (M+H)⁺.

### Step 4: Preparation of (S)—N-[3-(1-isopropyl-2-oxo-2,3-dihydro-1H-indol-5-yl)-2-oxo-oxazolidin-5-ylmethyl]-acetamide

Acetic anhydride (0.93 ml, 0.96 mmol) is added to (R)-(5-aminomethyl-2-oxo-oxazolidin-3-yl)-1-isopropyl-1,3-dihydro-indole-2-one (0.258 g, 0.639 mmol) and diisopropylethylamine (0.44 ml, 2.55 mmol) in dichloromethane (6 ml) at 0° C. The reaction is stirred at 0° C. for 30 minutes and then allowed to warm to room temperature. The mixture is diluted with dichloromethane, washed with water, citric acid and brine, dried (Na₂SO₄) and evaporated to give the title compound as an light brown solid. HPLC r.t. 3.597 min; ¹H NMR (300 MHz, CDCl₃) 7.54 (s, 1H), 7.24 (dd, J=2.4, 8.7 Hz, 1H), 6.97 (d, J=8.7 Hz, 1H), 6.12 (t, J=6 Hz, 1H), 4.73-4.82 (m, 1H), 4.61-4.71 (m, 1H), 4.04 (t, J=8.7 Hz, 1H), 3.74 (dd, J=6.6, 9 Hz, 1H), 3.57-3.75 (m, 2H), 3.51 (s, 2H), 2.03 (s, 3H), 1.46 (d, J=7.5 Hz, 6H); MS for C₁₇H₂₁N₃O₄ m/z 332 (M+H)⁺.

### Example 14

### Preparation of (S)—N-[3-(1-isopropyl-2-oxo-2,3-dihydro-1H-indol-5-yl)-2-oxo-oxazolidin-5-ylmethyl]-propionamide

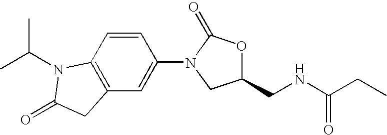

Propionic anhydride (0.047 ml, 0.371 mmol) is added dropwise to (R)-(5-aminomethyl-2-oxo-oxazolidin-3-yl)-1-isopropyl-1,3-dihydro-indole-2-one (preparation 4, 0.075 g, 0.185 mmol) and diisopropylethylamine (0.126 ml, 0.74 mmol) in dichloromethane (3 ml) at 0° C. The reaction is allowed to warm to room temperature and stirred for 1 hour. The mixture is diluted with dichloromethane, washed with water, citric acid and brine, dried (Na₂SO₄) and evaporated. The residue is purified by PTLC (10% MeOH/DCM) to give the title compound as a light brown solid. HPLC r.t. 3.767 min; ¹H NMR (300 MHz, CDCl₃) 7.54 (s, 1H), 7.24 (dd, J=2.4, 8.7 Hz, 1H), 6.97 (d, J=8.4 Hz, 1H), 5.94 (m, 1H), 4.73-4.80 (m, 1H), 4.61-4.70 (m, 1H), 4.03 (t, J=8.7 Hz, 1H), 3.80 (dd, J=6.6, 9 Hz, 1H), 3.58-3.72 (m, 2H), 3.50 (s, 2H), 2.24 (q, J=7.5 Hz, 2H), 1.45 (d, J=6.9 Hz, 6H), 1.14 (t, J=7.5 Hz, 3H); MS for C₁₈H₂₃N₃O₄ m/z 346 (M+H)⁺.

### Example 15

### Preparation of (S)—N-[3-(7-fluoro-1-isopropyl-2-oxo-2,3-dihydro-1H-indol-5-yl)-2-oxo-oxazolidin-5-ylmethyl]-acetamide

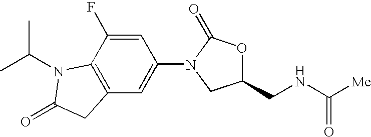

### Step 1: Preparation of (2,3-difluoro-5-nitrophenyl)acetic acid

(2,3-Difluoro-phenyl)-acetic acid (5 g, 0.0290 mol) is dissolved in concentrated sulfuric acid (20 ml) and the resulting solution cooled to −10° C. with vigorous stirring. A solution of nitric acid (1.88 ml, 69.3%, 0.0290 mol) and sulfuric acid (2 ml) is added dropwise at a rate such that the temperature remains below −5° C. The thickened slurry is stirred for 15 minutes and then poured on ice. The resulting white precipitate is filtered and dried under vacuum and consists of a 50/50 mixture of 5 and 6-NO₂ regioisomers suitable for use directly in the next step.

### Step 2: Preparation of 7-fluoro-1-isopropyl-5-nitro-1,3-dihydro-indol-2-one

Crude (2,3-difluoro-5-nitrophenyl)acetic acid (1.00 g, 0.00461 mol) and isopropylamine (6 eq., 2.35 ml, 0.0276 mol) are mixed in DMSO (5 ml) and stirred at 45° C. for 1 hour. 2N Hydrochloric acid (20 ml) is added in one portion and the mixture stirred at room temperature for 1 hour. The resulting light yellow precipitate is filtered, washed with water and ether, and dried under vacuum to give the title compound. HPLC r.t. 5.31 min.

### Step 3: Preparation of 5-amino-7-fluoro-1-isopropyl-1,3-dihydro-indol-2-one

Iron powder (0.854 g, 15.3 mmol) is added portionwise to a mixture of 7-fluoro-1-isopropyl-5-nitro-1,3-dihydro-indol-2-one (0.91 g, 3.82 mmol) and ammonium chloride (2.04 g, 38.2 mmol) in ethanol (50 ml) and water (25 ml) at 90° C. The reaction is stirred vigorously and heated for 30 min, cooled to room temperature and diluted with dichloromethane (150 ml). The mixture is filtered through celite, the organic layer separated and washed with water and brine, dried over sodium sulfate and evaporated to give the title compound as a dark brown gummy solid. HPLC r.t. 2.97 min; MS for C₁₁H₁₃FN₂O m/z 209.1 (M+H)⁺.

### Step 4: Preparation of (R)-[3-(7-fluoro-1-isopropyl-2-oxo-2,3-dihydro-1H-indol-5-ylamino)-2-hydroxy-propyl]-carbamic acid tert-butyl ester

5-Amino-7-fluoro-1-isopropyl-1,3-dihydro-indol-2-one (2.33 g, 11.2 mmol), (S)-oxiranylmethyl-carbamic acid tert-butyl ester (1.94 g, 11.2 mmol) and lithium trifluoromethanesulfonate (1.73 g, 11.2 mmol) in acetonitrile (20 ml) are heated at 70° C. for 2 hours. The reaction mixture is diluted with ethyl acetate, washed with water and brine, dried (Na₂SO₄) and evaporated. Final purification by flash chromatography (70% ethyl acetate/hexane) gives the title compound as a light brown foamy solid. HPLC r.t. 4.32 min; MS for C₁₉H₂₈FN₃O₄ m/z 382.3 (M+H)⁺.

### Step 5: Preparation of (S)-[3-(7-fluoro-1-isopropyl-2-oxo-2,3-dihydro-1H-indol-5-yl)-2-oxo-oxazolidin-5-ylmethyl]-carbamic acid tert-butyl ester

Phosgene (20% solution in toluene, 3.53 ml, 36.2 mmol) is added to (R)-[3-(7-fluoro-1-isopropyl-2-oxo-2,3-dihydro-1H-indol-5-ylamino)-2-hydroxy-propyl]-carbamic acid tert-butyl ester (Step 4, 1.84 g, 4.82 mmol) and triethylamine (3.33 ml, 24.1 mmol) in dichloromethane (20 ml) at 0° C. The reaction is allowed to warm to room temperature and stirred for 1 hours. The mixture is diluted with dichloromethane, washed with water and brine, dried (Na₂SO₄) and evaporated to give the title compound suitable for use directly in the next step (1.95 g, 99%); HPLC r.t. 5.28 min; MS for C₂₀H₂₆FN₃O₅ m/z 408 (M+H)⁺.

### Step 6: Preparation of (R)-5-(R-aminomethyl-2-oxo-oxazolidin-3-yl)-7-fluoro-1-isopropyl-1,3-dihydro-indol-2-one

(S)-[3-(7-Fluoro-1-isopropyl-2-oxo-2,3-dihydro-1H-indol-5-yl)-2-oxo-oxazolidin-5-ylmethyl]-carbamic acid tert-butyl ester (Step 5, 0.345 g, 0.665 mmol) is treated with 50% TFA/DCM (4 ml) for 30 minutes at room temperature. The reaction is evaporated and the title compound isolated as the TFA salt. HPLC r.t. 3.43 min; MS for C₁₅H₁₈FN₃O₃ m/z 308 (M+H)⁺.

### Step 7: Preparation of (S)—N-[3-(7-fluoro-1-isopropyl-2-oxo-2,3-dihydro-1H-indol-5-yl)-2-oxo-oxazolidin-5-ylmethyl]-acetamide

Acetic anhydride (0.671 ml, 7.12 mmol) is added to (R)-5-(5-aminomethyl-2-oxo-oxazolidin-3-yl)-7-fluoro-1-isopropyl-1,3-dihydro-indol-2-one (2.00 g, 4.74 mmol) and N,N-diisopropylethylamine (3.30 ml, 19.0 mmol) in dichloromethane (20 ml) at 0° C. The reaction is stirred at 0° C. for 30 minutes and then allowed to warm at room temperature. The mixture is diluted with dichloromethane, washed with water and brine, dried (Na₂SO₄) and evaporated. The residue is purified by PTLC (2% MeOH/DCM) to give the title compound as an off white solid (1.15 g, 70%); HPLC r.t. 4.07 min; ¹H NMR (300 MHz, CDCl₃) 7.31 (m, 1H), 7.23 (m, 1H), 5.97 (t, 1H), 4.86 (m, 1H), 4.78 (m, 1H), 4.02 (t, J=9 Hz, 1H), 3.77 (dd, J=6.6, 9 Hz, 1H), 3.58-3.73 (m, 2H), 3.55 (s, 2H), 2.03 (s, 3H), 1.42 (dd, J=1.5, 7.2 Hz, 6H); MS for C₁₇H₂₀FN₃O₄ m/z 350.0 (M+H)⁺.

### Example 16

### Preparation of (S)—N-[3-(7-fluoro-1-isopropyl-2-oxo-2,3-dihydro-1H-indol-5-yl)-2-oxo-oxazolidin-5-ylmethyl]-propionamide

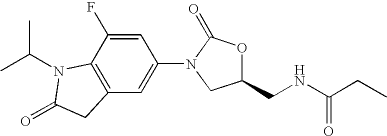

Propionic anhydride (0.088 ml, 0.688 mmol) is added dropwise to (R)-5-(5-aminomethyl-2-oxo-oxazolidin-3-yl)-7-fluoro-1-isopropyl-1,3-dihydro-indol-2-one (Example 16, 0.145 g, 0.344 mmol) and N,N-diisopropylethylamine (0.239 ml, 1.37 mmol) in dichloromethane (5 ml) at 0° C. The reaction is stirred at room temperature for 1 hour, then diluted with dichloromethane, washed with water and brine, dried (Na₂SO₄), and evaporated. The residue is purified by PTLC (10% methanol/dichloromethane) to give the title compound as a light yellow solid. HPLC r.t. 4.18 min; ¹H NMR (300 MHz, CDCl₃) 7.29 (m, 1H), 7.22 (dd, J=1.8, 15.9 Hz, 1H), 5.93 (t, 1H), 4.86 (m, 1H), 4.78 (m, 1H), 4.02 (t, J=9 Hz, 1H), 3.78 (dd, J=6.6, 9 Hz, 1H), 3.60-3.75 (m, 2H), 3.55 (s, 2H), 1.42 (dd, J=1.5, 6.9 Hz, 6H), 1.14 (t, J=7.5 Hz, 3H); MS for C₁₉H₂₂FN₃O₄ m/z 364.0 (M+H)⁺.

### Example 17

### Preparation of (S)—N-[2-oxo-3-(2-oxo-1-propyl-2,3-dihydro-1H-indol-5-yl)-oxazolidin-5-ylmethyl]-acetamide

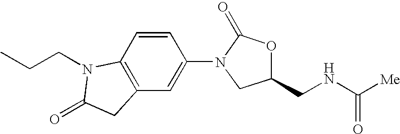

### Step 1: Preparation of (R)-[2-hydroxy-3-(2-oxo-1-propyl-2,3-dihydro-1H-indol-5-ylamino)-propyl]-carbamic acid tert-butyl ester

5-Amino-1-propyl-1,3-dihydro-indol-2-one (Preparation 5, 0.530 g, 2.78 mmol), (S)-oxiranylmethyl-carbamic acid tert-butyl ester (0.483 g, 2.78 mmol) and lithium trifluoromethanesulfonate (0.428 g, 2.78 mmol) in acetonitrile (7 ml) are heated at 90° C. for 1 hour. The reaction mixture is diluted with ethyl acetate, washed with water and brine, dried (Na₂SO₄) and evaporated. Final purification by flash chromatography (70% ethyl acetate/hexane) gives the title compound as a light brown solid. HPLC r.t. 3.66 min; MS for C19H29N3O4 m/z 364 (M+H)⁺.

### Step 2: Preparation of (S)-[2-oxo-3-(2-oxo-1-propyl-2,3-dihydro-1H-indol-5-yl)-oxazolidin-5-ylmethyl]-carbamic acid tert-butyl ester

Phosgene (20% solution in toluene, 0.978 ml, 10.0 mmol) is added to (R)-[2-hydroxy-3-(2-oxo-1-propyl-2,3-dihydro-1H-indol-5-ylamino)-propyl]-carbamic acid tert-butyl ester (Step 1, 0.520 g, 1.43 mmol) and triethylamine (0.998 ml, 7.15 mmol) in dichloromethane (10 ml) at 0° C. and stirred for 30 minutes. The mixture is diluted with dichloromethane, washed with water and brine, dried (Na₂SO₄) and evaporated to give the title compound as a light brown solid. HPLC r.t. 4.88 min; MS for C20H27N3O5 m/z 390 (M+H)⁺.

### Step 3: Preparation of (R)-5-(5-aminomethyl-2-oxo-oxazolidin-3-yl)-1-propyl-1,3-dihydro-indol-2-one

(S)-[2-Oxo-3-(2-oxo-1-propyl-2,3-dihydro-1H-indol-5-yl)-oxazolidin-5-ylmethyl]-carbamic acid tert-butyl ester (Step 2, 0.220 g, 0.564 mmol) is treated with 50% TFA/DCM (4 ml) for 15 minutes at room temperature. The reaction is evaporated and the title compound isolated as the TFA salt. HPLC r.t. 3.05 min; MS for C15H19N3O3 m/z 290.2 (M+H)⁺.

### Step 4: Preparation of (S)—N-[2-oxo-3-(2-oxo-1-propyl-2,3-dihydro-1H-indol-5-yl)-oxazolidin-5-ylmethyl]-acetamide

Acetic anhydride (0.082 ml, 0.848 mmol) is added to (R)-5-(5-Aminomethyl-2-oxo-oxazolidin-3-yl)-1-propyl-1,3-dihydro-indol-2-one (Step 3, 0.228 g, 0.565 mmol) and N,N-diisopropylethylamine (0.391 ml, 2.26 mmol) in dichloromethane (5 ml) at 0° C. The reaction is stirred at 0° C. for 60 minutes and then allowed to warm at room temperature. The mixture is diluted with dichloromethane, washed with water and brine, dried (Na₂SO₄) and evaporated. The residue is purified by PTLC (10% MeOH/DCM) to give the title compound as an off white solid. HPLC r.t. 3.65 min; ¹H NMR (300 MHz, CDCl₃) 7.56 (m, 1H), 7.24-7.28 (m, 1H), 6.80 (d, J=8.7 Hz, 1H), 6.01 (t, 1H), 4.74-4.82 (m, 1H), 4.05 (t, J=9 Hz, 1H), 3.80 (dd, J=6.6, 9 Hz, 2H), 3.57-3.76 (m, 4H), 3.54 (s, 2H), 2.03 (s, 3H), 1.64-1.74 (m, 2H), 0.97 (t, J=7.5 Hz, 3H); MS for C₁₇H₂₁N₃O₄ m/z 332.1 (M+H)⁺.

### Example 18

### Preparation of (S)—N-[2-oxo-3-(2-oxo-1-propyl-2,3-dihydro-1H-indol-5-yl)-oxazolidin-5-ylmethyl]-propionamide

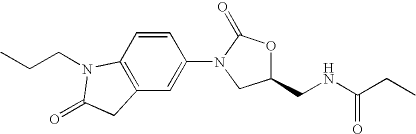

Propionic anhydride (0.089 ml, 0.694 mmol) is added dropwise to (R)-5-(5-Aminomethyl-2-oxo-oxazolidin-3-yl)-1-propyl-1,3-dihydro-indol-2-one (Example 17, 0.140 g, 0.347 mmol) and diisopropylethylamine (0.340 ml, 1.39 mmol) in dichloromethane (3 ml) at 0° C. The reaction is stirred at room temperature for 1 hour, then diluted with dichloromethane, washed with water and brine, dried (Na₂SO₄), and evaporated. The residue is purified by PTLC (5% MeOH/DCM) to give the title compound as a yellow solid (0.050 g, 67%); HPLC r.t. 3.79 min; ¹H NMR (300 MHz, CDCl₃) δ 7.56 (m, 1H), 7.23-7.27 (m, 1H), 6.80 (d, J=8.4 Hz, 1H), 5.92 (t, 1H), 4.77 (m, 1H), 4.04 (t, J=8.7 Hz, 1H), 3.80 (dd, J=6.6, 9 Hz, 2H), 3.59-3.75 (m, 4H), 3.54 (s, 2H), 2.25 (q, J=7.8 Hz, 2H), 1.69 (m, 3H), 1.14 (t, J=7.5 Hz, 3H), 0.96 (t, J=7.5 Hz, 3H); MS for C₁₈H₂₃N₃O₄ m/z 346.2 (M+H)⁺.

### Example 19

### Preparation of (S)—N-[3-(7-fluoro-2-oxo-1-propyl-2,3-dihydro-1H-indol-5-yl)-2-oxo-oxazolidin-5-ylmethyl]-acetamide

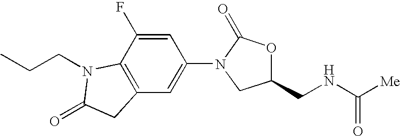

### Step 1: Preparation of (R)-[3-(7-fluoro-2-oxo-1-propyl-2,3-dihydro-1H-indol-5-ylamino)-2-hydroxy-propyl]-carbamic acid tert-butyl ester

5-Amino-7-fluoro-1-propyl-1,3-dihydro-indol-2-one (Preparation 6, 0.500 g, 2.40 mmol), (S)-oxiranylmethyl-carbamic acid tert-butyl ester (0.417 g, 2.40 mmol) and lithium trifluoromethanesulfonate (0.368 g, 2.40 mmol) in acetonitrile (8 ml) are heated at 90° C. for 8 hours. The reaction mixture is diluted with ethyl acetate, washed with water and brine, dried (Na₂SO₄) and evaporated. Final purification by flash chromatography (70% Ethyl acetate/hexane) gives pure the title compound as a yellow solid. HPLC r.t. 4.30 min; MS for C19H28FN3O4 m/z 382.3 (M+H)⁺.

### Step 2: Preparation of (S)-[3-(7-fluoro-2-oxo-1-propyl-2,3-dihydro-1H-indol-5-yl)-2-oxo-oxazolidin-5-ylmethyl]-carbamic acid tert-butyl ester

Phosgene (20% solution in toluene, 0.770 ml, 7.86 mmol) is added to (R)-[3-(7-Fluoro-2-oxo-1-propyl-2,3-dihydro-1H-indol-5-ylamino)-2-hydroxy-propyl]-carbamic acid tert-butyl ester (Step 1, 0.400 g, 1.05 mmol) and triethylamine (0.726 ml, 5.24 mmol) in dichloromethane (5 ml) at 0° C. and stirred for 30 minutes. The mixture is diluted with dichloromethane, washed with water and brine, dried (Na₂SO₄) and evaporated to give the title compound as a dark yellow solid. HPLC r.t. 5.28 min; MS for C20H26FN3O5 m/z 408.1 (M+H)⁺.

### Step 3: Preparation of (R)-5-(5-aminomethyl-2-oxo-oxazolidin-3-yl)-7-fluoro-1-propyl-1,3-dihydro-indol-2-one

(S)-[3-(7-Fluoro-2-oxo-1-propyl-2,3-dihydro-1H-indol-5-yl)-2-oxo-oxazolidin-5-ylmethyl]-carbamic acid tert-butyl ester (0.20 g, 0.491 mmol) is treated with 50% TFA/DCM (4 ml) for 15 minutes at room temperature. The reaction is evaporated and the product isolated as the TFA salt. HPLC r.t. 3.43 min; MS for C15H18FN3O3 Mol. Wt.: 307.32 m/z 308.0 (M+H)⁺.

### Step 4: Preparation of (S)—N-[3-(7-fluoro-2-oxo-1-propyl-2,3-dihydro-1H-indol-5-yl)-2-oxo-oxazolidin-5-ylmethyl]-acetamide

Acetic anhydride (0.069 ml, 0.733 mmol) is added to (R)-5-(5-Aminomethyl-2-oxo-oxazolidin-3-yl)-7-fluoro-1-propyl-1,3-dihydro-indol-2-one (0.206 g, 0.489 mmol) and N,N-diisopropylethylamine (0.358 ml, 1.96 mmol) in dichloromethane (4 ml) at 0° C. The reaction is stirred at 0° C. for 30 minutes and then allowed to warm at room temperature. The mixture is diluted with dichloromethane, washed with water and brine, dried (Na₂SO₄) and evaporated. The residue is purified by PTLC (5% MeOH/DCM) to give the title compound as an off white solid. HPLC r.t. 4.01 min; ¹H NMR (300 MHz, CDCl₃) 7.24 (m, 1H), 7.19 (m, 1H), 6.04 (t, 1H), 4.78 (m, 1H), 4.02 (t, J=9 Hz, 1H), 3.72-3.81 (m, 3H), 3.59-3.72 (m, 2H), 3.56 (s, 2H), 2.03 (s, 3H), 1.69 (m, 2H), 0.95 (t, J=7.5 Hz, 3H); MS for C₁₇H₂₀FN₃O₄ m/z 350.0 (M+H)⁺.

### Example 20

### Preparation of (S)—N-[3-(7-Fluoro-2-oxo-1-propyl-2,3-dihydro-1H-indol-5-yl)-2-oxo-oxazolidin-5-ylmethyl]-propionamide

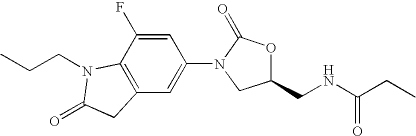

Propionic anhydride (0.066 ml, 0.515 mmol) is added dropwise to (R)-5-(5-minomethyl-2-oxo-oxazolidin-3-yl)-7-fluoro-1-propyl-1,3-dihydro-indol-2-one (Example 19, 0.108 g, 0.257 mmol) and diisopropylethylamine (0.176 ml, 1.03 mmol) in dichloromethane (3 ml) at 0° C. The reaction is stirred at room temperature for 1 hour, then diluted with dichloromethane, washed with water and brine, dried (Na₂SO₄), and evaporated. The residue is purified by PTLC (5% MeOH/DCM) to give the title compound as a yellow solid. HPLC r.t. 4.23 min; ¹H NMR (300 MHz, CDCl₃) 7.30 (m, 1H), 7.24 (m, 1H), 7.19 (m, 1H), 5.94 (t, 1H), 4.78 (m, 1H), 4.01 (t, J=9 Hz, 1H), 3.78 (m, 3H), 3.59-3.78 (m, 2H), 3.56 (s, 2H), 2.24 (q, J=7.5 Hz, 2H), 1.69 (m, 2H), 1.14 (t, J=7.5 Hz, 3H), 0.95 (t, J=7.5 Hz, 3H); MS for C₁₈H₂₂FN₃O₄ m/z 364.0 (M+H)⁺.

### Example 21

### Preparation of (S)—N-[3-(1-cyclopropyl-2-oxo-2,3-dihydro-1H-indol-5-yl)-2-oxo-oxazolidin-5-ylmethyl]-acetamide

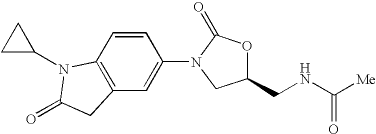

### Step 1: Preparation of (R)-[3-(1-cyclopropyl-2-oxo-2,3-dihydro-1H-indol-5-ylamino)-2-hydroxy-propyl]-carbamic acid tert-butyl ester

5-Amino-1-cyclopropyl-1,3-dihydro-indol-2-one (Preparation 7, 1.30 g, 6.90 mmol), (S)-oxiranylmethyl-carbamic acid tert-butyl ester (1.20 g, 6.90 mmol) and lithium trifluoromethanesulfonate (1.06 g, 6.90 mmol) in acetonitrile (10 ml) are heated at 90° C. for 3 hours. The reaction mixture is diluted with ethyl acetate, washed with water and brine, dried (Na₂SO₄) and evaporated. Final purification by flash chromatography (70% ethyl acetate/hexane) gives pure the title compound as a light brown solid. HPLC r.t. 3.48 min; MS for C19H27N3O4 m/z 362.3 (M+H)⁺.

### Step 2: Preparation of (S)-[3-(1-cyclopropyl-2-oxo-2,3-dihydro-1H-indol-5-yl)-2-oxo-oxazolidin-5-ylmethyl]-carbamic acid tert-butyl ester

Phosgene (20% solution in toluene, 1.95 ml, 19.9 mmol) is added to (R)-[3-(1-cyclopropyl-2-oxo-2,3-dihydro-1H-indol-5-ylamino)-2-hydroxy-propyl]-carbamic acid tert-butyl ester (Step 1, 0.720 g, 1.99 mmol) and triethylamine (1.38 ml, 9.96 mmol) in dichloromethane (10 ml) at 0° C. and stirred for 30 minutes. The mixture is diluted with dichloromethane, washed with water and brine, dried (Na₂SO₄) and evaporated. The residue is purified by flash column chromatography (70% ethyl acetate/hexane) to give the title compound as an off-white solid (0.58 g, 75%); HPLC r.t. 4.63 min; MS for C20H25N3O5 m/z 388.2 (M+H)⁺.

### Step 3: Preparation of (R)-5-(5-aminomethyl-2-oxo-oxazolidin-3-yl)-1-cyclopropyl-1,3-dihydro-indol-2-one

(S)-[3-(1-Cyclopropyl-2-oxo-2,3-dihydro-1H-indol-5-yl)-2-oxo-oxazolidin-5-ylmethyl]-carbamic acid tert-butyl ester (Step 2, 0.240 g, 0.619 mmol) is treated with 50% TFA/DCM (4 ml) for 15 minutes at room temperature. The reaction is evaporated and the title compound isolated as the TFA salt. HPLC r.t. 2.81 min; MS for C15H17N3O3 m/z 288.1 (M+H)⁺.

### Step 4: Preparation of (S)—N-[3-(1-cyclopropyl-2-oxo-2,3-dihydro-1H-indol-5-yl)-2-oxo-oxazolidin-5-ylmethyl]-acetamide

Acetic anhydride (0.092 ml, 0.971 mmol) is added to 5-(5-Aminomethyl-2-oxo-oxazolidin-3-yl)-1-cyclopropyl-1,3-dihydro-indol-2-one (Step 3, 0.260 g, 0.647 mmol) and N,N-diisopropylethylamine (0.473 ml, 2.58 mmol) in dichloromethane (5 ml) at 0° C. The reaction is stirred at 0° C. for 60 minutes and then allowed to warm at room temperature. The mixture is diluted with dichloromethane, washed with water and brine, dried (Na₂SO₄) and evaporated. The residue is purified by PTLC (10% MeOH/DCM) to give the title compound as an off white solid. HPLC r.t. 3.36 min; ¹H NMR (300 MHz, CDCl₃) 7.52 (m, 1H), 7.24-7.27 (m, 1H), 7.05 (dd, J=2.4, 8.7 Hz, 1H), 6.11 (t, 1H), 4.76 (m, 1H), 4.04 (t, J=9 Hz, 1H), 3.79 (dd, J=6.3, 9 Hz, 1H), 3.57-3.74 (m, 2H), 3.49 (s, 2H), 2.63 (m, 1H), 2.03 (s, 3H), 1.07 (m, 2H), 0.89 (m, 2H); MS for C₁₇H₁₉N₃O₄ m/z 330.1 (M+H)⁺.

### Example 22

### Preparation of (S)—N-[3-(1-cyclopropyl-2-oxo-2,3-dihydro-1H-indol-5-yl)-2-oxo-oxazolidin-5-ylmethyl]-propionamide

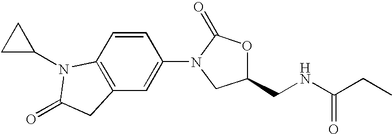

Propionic anhydride (0.099 ml, 0.774 mmol) is added dropwise to (R)-5-(5-aminomethyl-2-oxo-oxazolidin-3-yl)-1-cyclopropyl-1,3-dihydro-indol-2-one (Example 21, 0.155 g, 0.387 mmol) and diisopropylethylamine (0.265 ml, 1.55 mmol) in dichloromethane (4 ml) at 0° C. The reaction is stirred at room temperature for 1 hour, then diluted with dichloromethane, washed with water and brine, dried (Na₂SO₄), and evaporated. The residue is purified by PTLC (5% MeOH/DCM) to give the title compound as a yellow solid. HPLC r.t. 3.55 min; ¹H NMR (300 MHz, CDCl₃) 7.53 (m, 1H), 7.25 (m, 1H), 7.05 (d, J=8.7 Hz, 1H), 6.00 (t, J=6 Hz, 1H), 4.77 (m, 1H), 4.04 (t, J=8.7 Hz, 1H), 3.80 (dd, J=6.6, 9.3 Hz, 1H), 3.59-3.77 (m, 2H), 3.49 (s, 2H), 2.63 (m, 1H), 2.25 (q, J=7.5 Hz, 2H), 1.14 (t, J=7.5 Hz, 3H), 1.07 (m, 2H), 0.89 (m, 2H); MS for C₁₈H₂₁N₃O₄ m/z 344.1 (M+H)⁺.

### Example 23

### Preparation of (S)—N-[3-(1-cyclopropyl-7-fluoro-2-oxo-2,3-dihydro-1H-indol-5-yl)-2-oxo-oxazolidin-5-ylmethyl]-acetamide

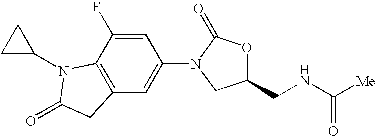

### Step 1: Preparation of 1-cyclopropyl-7-fluoro-5-nitro-1,3-dihydro-indol-2-one

Crude (2,3-difluoro-5-nitrophenyl)acetic acid (Example 15, 2.50 g, 11.5 mmol) and cyclopropylamine (6 eq., 4.78 ml, 0.0691 mol) are mixed in DMSO (10 ml) and stirred at 50° C. for 20 hours. 2N Hydrochloric acid (40 ml) is added in one portion and the mixture stirred at room temperature for 2 hours. The resulting light yellow precipitate is filtered, washed with water and dried under vacuum. The residue is purified by flash column chromatography (20% Ethyl acetate/hexane) to give the title compound as a yellow solid. HPLC r.t. 4.90 min; MS for C11H9FN2O3 m/z 235.0 (M−H)⁻.

### Step 2: Preparation of 5-amino-1-cyclopropyl-7-fluoro-1,3-dihydro-indol-2-one

Iron powder (0.994 g, 17.8 mmol) is added in small portions to 1-cyclopropyl-7-fluoro-5-nitro-1,3-dihydro-indol-2-one (1.05 g, 4.44 mmol) and ammonium chloride (2.32 g, 44.5 mmol) in ethanol (50 ml) and water (25 ml) at 90° C. The reaction mixture is stirred vigorously and heated for 60 min, cooled to room temperature, and diluted with dichloromethane (250 ml). The mixture is filtered through celite, the organic layer separated and washed with water and brine, dried over sodium sulfate and evaporated to give the title compound as a yellowish-brown solid. HPLC r.t. 2.62 min; MS for C11H11FN2O m/z 207.2 (M+H)⁺.

### Step 3: Preparation of (R)-[3-(1-cyclopropyl-7-fluoro-2-oxo-2,3-dihydro-1H-indol-5-ylamino)-2-hydroxy-propyl]-carbamic acid tert-butyl ester

5-Amino-1-cyclopropyl-7-fluoro-1,3-dihydro-indol-2-one (0.560 g, 2.72 mmol), (S)-oxiranylmethyl-carbamic acid tert-butyl ester (0.472 g, 2.72 mmol) and lithium trifluoromethanesulfonate (0.415 g, 2.72 mmol) in acetonitrile (5 ml) are heated at 85° C. for 4.5 hours. The reaction mixture is diluted with ethyl acetate, washed with water and brine, dried (Na₂SO₄) and evaporated. Final purification by flash chromatography (70% Ethyl acetate/hexane) gives the title compound as a yellow solid. HPLC r.t. 3.98 min; MS for C19H26FN3O4 m/z 380.1 (M+H)⁺.

### Step 4: Preparation of (S)-[3-(1-cyclopropyl-7-fluoro-2-oxo-2,3-dihydro-1H-indol-5-yl)-2-oxo-oxazolidin-5-ylmethyl]-carbamic acid tert-butyl ester

Phosgene (20% solution in toluene, 0.754 ml, 7.71 mmol) is added to (R)-[3-(1-cyclopropyl-7-fluoro-2-oxo-2,3-dihydro-1H-indol-5-ylamino)-2-hydroxy-propyl]-carbamic acid tert-butyl ester (Step 3, 0.390 g, 1.03 mmol) and triethylamine (0.711 ml, 5.13 mmol) in dichloromethane (5 ml) at 0° C. and stirred for 30 minutes. The mixture is diluted with dichloromethane, washed with water and brine, dried (Na₂SO₄) and evaporated to give the title compound as a yellow solid. HPLC r.t. 4.96 min; MS for C20H24FN3O5 m/z 406 (M+H)⁺.

### Step 5: Preparation of (R)-5-(5-aminomethyl-2-oxo-oxazolidin-3-yl)-1-cyclopropyl-7-fluoro-1,3-dihydro-indol-2-one

(S)-[3-(1-Cyclopropyl-7-fluoro-2-oxo-2,3-dihydro-1H-indol-5-yl)-2-oxo-oxazolidin-5-ylmethyl]-carbamic acid tert-butyl ester (0.20 g, 0.493 mmol) is treated with 50% TFA/DCM (4 ml) for 15 minutes at room temperature. The reaction is evaporated and the title compound isolated as the TFA salt. HPLC r.t. 3.17 min; MS for C15H16FN3O3 m/z 306.3 (M+H)⁺.

### Step 6: Preparation of (S)—N-[3-(1-cyclopropyl-7-fluoro-2-oxo-2,3-dihydro-1H-indol-5-yl)-2-oxo-oxazolidin-5-ylmethyl]-acetamide

Acetic anhydride (0.070 ml, 0.740 mmol) is added to (R)-5-(5-aminomethyl-2-oxo-oxazolidin-3-yl)-1-cyclopropyl-7-fluoro-1,3-dihydro-indol-2-one (0.207 g, 0.493 mmol) and N,N-diisopropylethylamine (0.344 ml, 1.97 mmol) in dichloromethane (4 ml) at 0° C. The reaction is stirred at 0° C. for 30 minutes and then allowed to warm at room temperature. The mixture is diluted with dichloromethane, washed with water and brine, dried (Na₂SO₄) and evaporated. The residue is purified by PTLC (5% MeOH/DCM) to give the title compound as an off white solid (0.125 g, 73%); HPLC r.t. 3.70 min; ¹H NMR (300 MHz, CDCl₃) 7.24 (m, 1H), 7.19 (m, 1H), 6.04 (t, 1H), 4.78 (m, 1H), 4.02 (t, J=8.7 Hz, 1H), 3.76 (dd, J=6.6, 9 Hz, 1H), 3.58-3.70 (m, 2H), 3.51 (s, 2H), 2.84 (m, 1H), 2.03 (s, 3H), 1.05 (m, 2H), 0.96 (m, 2H); MS for C₁₇H₁₈FN₃O₄ m/z 348 (M+H)⁺.

### Example 24

### Preparation of (S)—N-[3-(1-sec-butyl-2-oxo-2,3-dihydro-1H-indol-5-yl)-2-oxo-oxazolidin-5-ylmethyl]-acetamide

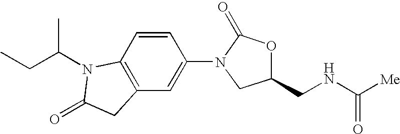

### Step 1: Preparation of (R)-[3-(1-sec-butyl-2-oxo-2,3-dihydro-1H-indol-5-ylamino)-2-hydroxy-propyl]-carbamic acid tert-butyl ester

5-Amino-1-sec-butyl-1,3-dihydro-indol-2-one (Preparation 8, 0.480 g, 2.35 mmol), (S)-oxiranylmethyl-carbamic acid tert-butyl ester (0.409 g, 2.35 mmol) and lithium trifluoromethanesulfonate (0.361 g, 2.35 mmol) in acetonitrile (5 ml) are heated at 90° C. for 5 hours. The reaction mixture is diluted with ethyl acetate, washed with water and brine, dried (Na₂SO₄) and evaporated. Final purification by flash chromatography (70% ethyl acetate/hexane) gives the title compound as a light brown foamy solid. HPLC r.t. 3.85 min; MS for C20H31N3O4 m/z 378.4 (M+H)⁺.

### Step 2: Preparation of (S)-[3-(1-sec-butyl-2-oxo-2,3-dihydro-1H-indol-5-yl)-2-oxo-oxazolidin-5-ylmethyl]-carbamic acid tert-butyl ester

Phosgene (20% solution in toluene, 0.854 ml, 8.74 mmol) is added to (R)-[3-(1-sec-butyl-2-oxo-2,3-dihydro-1H-indol-5-ylamino)-2-hydroxy-propyl]-carbamic acid tert-butyl ester (0.330 g, 0.874 mmol) and triethylamine (0.610 ml, 4.37 mmol) in dichloromethane (5 ml) at 0° C. The reaction is allowed to warm to room temperature and stirred for 1 hours. The mixture is diluted with dichloromethane, washed with water and brine, dried (Na₂SO₄) and evaporated to give the title compound suitable for use directly in the next step. HPLC r.t. 5.15 min; MS for C21H29N3O5 m/z 404 (M+H)⁺.

### Step 3: Preparation of (R)-5-(5-aminomethyl-2-oxo-oxazolidin-3-yl)-1-sec-butyl-1,3-dihydro-indol-2-one

[3-(1-sec-Butyl-2-oxo-2,3-dihydro-1H-indol-5-yl)-2-oxo-oxazolidin-5-ylmethyl]-carbamic acid tert-butyl ester (0.345 g, 0.665 mmol) is treated with 50% TFA/DCM (4 ml) for 30 minutes at room temperature. The reaction is evaporated and the title compound isolated as the TFA salt. HPLC r.t. 3.32 min; MS for C16H21N3O3 m/z 304.2 (M+H)⁺.

### Step 4: Preparation of (S)—N-[3-(1-sec-butyl-2-oxo-2,3-dihydro-1H-indol-5-yl)-2-oxo-oxazolidin-5-ylmethyl]-acetamide

Acetic anhydride (0.070 ml, 0.718 mmol) is added to (R)-5-(5-aminomethyl-2-oxo-oxazolidin-3-yl)-1-sec-butyl-1,3-dihydro-indol-2-one (Step 3, 0.200 g, 0.479 mmol) and N,N-diisopropylethylamine (0.332 ml, 1.91 mmol) in dichloromethane (4 ml) at 0° C. The reaction is stirred at 0° C. for 30 minutes and then allowed to warm at room temperature. The mixture is diluted with dichloromethane, washed with water and brine, dried (Na₂SO₄) and evaporated. The residue is purified by PTLC (5% MeOH/DCM) to give the title compound as an off white solid. HPLC r.t. 3.87 min; ¹H NMR (300 MHz, CDCl₃) 7.54 (m, 1H), 7.23 (dd, J=2.4, 8.4 Hz, 1H), 6.95 (d, J=8.4 Hz, 1H), 5.98 (t, 1H), 4.77 (m, 1H), 4.39 (m, 1H), 4.04 (t, J=9 Hz, 1H), 3.78 (dd, J=6.9, 9.6 Hz, 1H), 3.56-3.75 (m, 3H), 3.53 (s, 2H), 2.03 (s, 3H), 1.91-2.01 (m, 1H), 1.77 (m, 1H), 1.44 (d, J=6.9 Hz, 3H), 0.87 (t, J=7.5 Hz, 3H); MS for C₁₈H₂₃N₃O₄ m/z 346.2 (M+H)⁺.

### Example 25

### Preparation of (S)—N-[3-(1-sec-butyl-2-oxo-2,3-dihydro-1H-indol-5-yl)-2-oxo-oxazolidin-5-ylmethyl]-propionamide

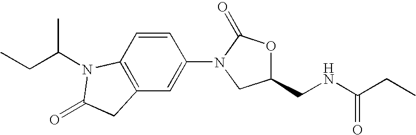

Propionic anhydride (0.104 ml, 0.814 mmol) is added dropwise to (R)-5-(5-aminomethyl-2-oxo-oxazolidin-3-yl)-1-sec-butyl-1,3-dihydro-indol-2-one (Example 24, 0.170 g, 0.407 mmol) and diisopropylethylamine (0.279 ml, 1.63 mmol) in dichloromethane (4 ml) at 0° C. The reaction is stirred at room temperature for 1 hour, then diluted with dichloromethane, washed with water and brine, dried (Na₂SO₄), and evaporated. The residue is purified by PTLC (5% MeOH/DCM) to give the title compound as a yellow solid. HPLC r.t. 4.07 min; ¹H NMR (300 MHz, CDCl₃) 7.54 (m, 1H), 7.22 (dd, J=2.7, 8.7 Hz, 1H), 6.95 (d, J=9 Hz, 1H), 5.93 (t, 1H), 4.77 (m, 1H), 4.39 (m, 1H), 4.03 (t, J=8.7 Hz, 1H), 3.79 (dd, J=6.6, 9 Hz, 1H), 3.58-3.75 (m, 3H), 3.53 (s, 2H), 2.25 (q, J=7.5 Hz, 2H), 1.91-2.01 (m, 1H), 1.77 (m, 1H), 1.44 (d, J=6.9 Hz, 3H), 1.15 (t, J=7.5 Hz, 3H), 0.87 (t, J=7.5 Hz, 3H); MS for C₁₉H₂₅N₃O₄ m/z 360.2 (M+H)⁺.

### Example 26

### Preparation of (S)—N-[3-(1-cyclopropylmethyl-2-oxo-2,3-dihydro-1H-indol-5-yl)-2-oxo-oxazolidin-5-ylmethyl]-acetamide

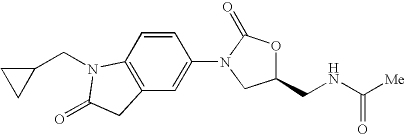

### Step 1: Preparation of (R)-[3-(1-cyclopropylmethyl-2-oxo-2,3-dihydro-1H-indol-5-ylamino)-2-hydroxy-propyl]-carbamic acid tert-butyl ester

5-Amino-1-cyclopropylmethyl-1,3-dihydro-indol-2-one (Preparation 7, 0.400 g, 1.98 mmol), (S)-oxiranylmethyl-carbamic acid tert-butyl ester (0.344 g, 1.98 mmol) and lithium trifluoromethanesulfonate (0.304 g, 1.98 mmol) in acetonitrile (5 ml) are heated at 90° C. for 4 hours. The reaction mixture is diluted with ethyl acetate, washed with water and brine, dried (Na₂SO₄) and evaporated. Final purification by flash chromatography (70% ethyl acetate/hexane) gives the title compound as a light brown foamy solid. HPLC r.t. 3.76 min; MS for C20H29N3O4 m/z 376.3 (M+H)⁺.

### Step 2: Preparation of (S)-[3-(1-cyclopropylmethyl-2-oxo-2,3-dihydro-1H-indol-5-yl)-2-oxo-oxazolidin-5-ylmethyl]-carbamic acid tert-butyl ester

Phosgene (20% solution in toluene, 0.728 ml, 7.45 mmol) is added to (R)-[3-(1-Cyclopropylmethyl-2-oxo-2,3-dihydro-1H-indol-5-ylamino)-2-hydroxy-propyl]-carbamic acid tert-butyl ester (0.280 g, 0.745 mmol) and triethylamine (0.520 ml, 3.73 mmol) in dichloromethane (5 ml) at 0° C. The reaction is allowed to warm to room temperature and stirred for 1 hours. The mixture is diluted with dichloromethane, washed with water and brine, dried (Na₂SO₄) and evaporated to give the title compound suitable for use directly in the next step. HPLC r.t. 5.01 min; MS for C21H27N3O5 m/z 402 (M+H)⁺.

### Step 3: Preparation of (R)-5-(5-aminomethyl-2-oxo-oxazolidin-3-yl)-1-cyclopropylmethyl-1,3-dihydro-indol-2-one

(S)-[3-(1-Cyclopropylmethyl-2-oxo-2,3-dihydro-1H-indol-5-yl)-2-oxo-oxazolidin-5-ylmethyl]-carbamic acid tert-butyl ester (0.210 g, 0.523 mmol) is treated with 50% TFA/DCM (4 ml) for 30 minutes at room temperature. The reaction is evaporated and the title compound isolated as the TFA salt. HPLC r.t. 3.21 min; MS for C16H19N3O3 m/z 302.1 (M+H)⁺.

### Step 4: Preparation of (S)—N-[3-(1-cyclopropylmethyl-2-oxo-2,3-dihydro-1H-indol-5-yl)-2-oxo-oxazolidin-5-ylmethyl]-acetamide

Acetic anhydride (0.076 ml, 0.784 mmol) is added to (R)-5-(5-Aminomethyl-2-oxo-oxazolidin-3-yl)-1-cyclopropylmethyl-1,3-dihydro-indol-2-one (0.217 g, 0.523 mmol) and N,N-diisopropylethylamine (0.362 ml, 2.09 mmol) in dichloromethane (4 ml) at 0° C. The reaction is stirred at 0° C. for 30 minutes and then allowed to warm at room temperature. The mixture is diluted with dichloromethane, washed with water and brine, dried (Na₂SO₄) and evaporated. The residue is purified by PTLC (5% MeOH/DCM) to give the title compound as an off white solid. HPLC r.t. 3.75 min; ¹H NMR (300 MHz, CDCl₃) 7.56 (s, 1H), 7.26 (m, 1H), 6.88 (d, J=8.4 Hz, 1H), 6.04 (t, J=6 Hz, 1H), 4.74-4.82 (m, 1H), 4.05 (t, J=8.7 Hz, 1H), 3.79 (dd, J=6.6, 9 Hz, 1H), 3.59-3.75 (m, 2H), 3.59 (d, J=6.9 Hz, 2H), 3.55 (s, 2H), 2.03 (s, 3H), 1.15 (m, 1H), 0.52 (m, 2H), 0.38 (m, 2H); MS for C₁₈H₂₁N₃O₄ m/z 344.1 (M+H)⁺.

### Example 27

### Preparation of (S)—N-[3-(1-cyclopropylmethyl-2-oxo-2,3-dihydro-1H-indol-5-yl)-2-oxo-oxazolidin-5-ylmethyl]-propionamide

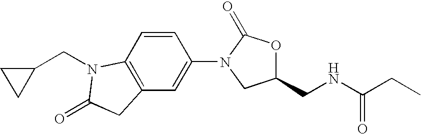

Propionic anhydride (0.095 ml, 0.746 mmol) is added dropwise to (R)-5-(5-aminomethyl-2-oxo-oxazolidin-3-yl)-1-cyclopropylmethyl-1,3-dihydro-indol-2-one (Example 26, 0.155 g, 0.373 mmol) and diisopropylethylamine (0.273 ml, 1.49 mmol) in dichloromethane (3 ml) at 0° C. The reaction is stirred at room temperature for 1 hour, then diluted with dichloromethane, washed with water and brine, dried (Na₂SO₄), and evaporated. The residue is purified by PTLC (5% MeOH/DCM) to give the title compound as an off-white solid. HPLC r.t. 3.93 min; ¹H NMR (300 MHz, CDCl₃) 7.56 (s, 1H), 7.27 (m, 1H), 6.88 (d, J=8.4 Hz, 1H), 5.95 (t, 1H), 4.73-4.82 (m, 1H), 4.04 (t, J=8.7 Hz, 1H), 3.80 (dd, J=6.6, 9 Hz, 1H), 3.59-3.77 (m, 2H), 3.59 (d, J=6.9 Hz, 2H), 3.55 (s, 2H), 2.25 (q, J=7.5 Hz, 2H), 1.14 (m, 1H), 0.53 (m, 2H), 0.38 (m, 2H); MS for C₁₉H₂₅N₃O₄ m/z 358.1 (M+H)⁺.

### Example 28

### Preparation of (S)—N-{3-[1-(2-fluoro-1-methyl-ethyl)-2-oxo-2,3-dihydro-1H-indol-5-yl]-2-oxo-oxazolidin-5-ylmethyl}-acetamide

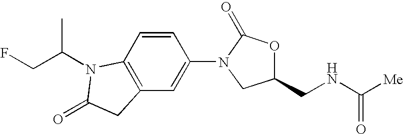

### Step 1: Preparation of (R)-{3-[1-(2-fluoro-1-methyl-ethyl)-2-oxo-2,3-dihydro-1H-indol-5-ylamino]-2-hydroxy-propyl}-carbamic acid tert-butyl ester

5-Amino-1-(2-fluoro-1-methyl-ethyl)-1,3-dihydro-indol-2-one (Preparation 10, 0.370 g, 1.77 mmol), (S)-oxiranylmethyl-carbamic acid tert-butyl ester (0.308 g, 1.77 mmol) and lithium trifluoromethanesulfonate (0.271 g, 1.77 mmol) in acetonitrile (5 ml) are heated at 90° C. for 3 hours. The reaction mixture is diluted with ethyl acetate, washed with water and brine, dried (Na₂SO₄) and evaporated. Final purification by PTLC (5% methanol/dichloromethane) gives the title compound as an off-white solid. HPLC r.t. 3.59 min; MS for C19H28FN3O4 m/z 382.3 (M+H)⁺.

### Step 2: Preparation of (S)-{3-[1-(2-fluoro-1-methyl-ethyl)-2-oxo-2,3-dihydro-1H-indol-5-yl]-2-oxo-oxazolidin-5-ylmethyl}-carbamic acid tert-butyl ester

Phosgene (20% solution in toluene, 0.635 ml, 6.48 mmol) is added to (R)-{3-[1-(2-Fluoro-1-methyl-ethyl)-2-oxo-2,3-dihydro-1H-indol-5-ylamino]-2-hydroxy-propyl}-carbamic acid tert-butyl ester (0.330 g, 0.865 mmol) and triethylamine (0.600 ml, 4.32 mmol) in dichloromethane (5 ml) at 0° C. The reaction is allowed to warm to room temperature and stirred for 1 hours. The mixture is diluted with dichloromethane, washed with water and brine, dried (Na₂SO₄) and evaporated to give the title compound suitable for use directly in the next step. HPLC r.t. 4.78 min; MS for C20H26FN3O5 m/z 408.1 (M+H)⁺.

### Step 3: Preparation of (R)-5-(5-aminomethyl-2-oxo-oxazolidin-3-yl)-1-(2-fluoro-1-methyl-ethyl)-1,3-dihydro-indol-2-one

(S)-{3-[1-(2-Fluoro-1-methyl-ethyl)-2-oxo-2,3-dihydro-1H-indol-5-yl]-2-oxo-oxazolidin-5-ylmethyl}-carbamic acid tert-butyl ester (0.250 g, 0.614 mmol) is treated with 50% TFA/DCM (4 ml) for 30 minutes at room temperature. The reaction is evaporated and the title compound isolated as the TFA salt. HPLC r.t. 2.95 min; MS for C15H18FN3O3 m/z 308.0 (M+H)⁺.

### Step 4: Preparation of (S)—N-{3-[1-(2-fluoro-1-methyl-ethyl)-2-oxo-2,3-dihydro-1H-indol-5-yl]-2-oxo-oxazolidin-5-ylmethyl}-acetamide

Acetic anhydride (0.084 ml, 0.889 mmol) is added to 5-(5-Aminomethyl-2-oxo-oxazolidin-3-yl)-1-(2-fluoro-1-methyl-ethyl)-1,3-dihydro-indol-2-one (0.250 g, 0.593 mmol) and N,N-diisopropylethylamine (0.435 ml, 2.37 mmol) in dichloromethane (5 ml) at 0° C. The reaction is stirred at 0° C. for 30 minutes and then allowed to warm at room temperature. The mixture is diluted with dichloromethane, washed with water and brine, dried (Na₂SO₄) and evaporated. The residue is purified by PTLC (5% MeOH/DCM) to give the title compound as a light brown solid. HPLC r.t. 3.53 min; ¹H NMR (300 MHz, CDCl₃) 7.57 (m, 1H), 7.23 (m, 1H), 6.97 (d, J=8.7 Hz, 1H), 6.04 (t, 1H), 4.93 (m, 1H), 4.52-4.78 (m, 3H), 4.04 (t, J=9 Hz, 1H), 3.78 (dd, J=6.6, 9 Hz, 1H), 3.57-3.75 (m, 2H), 3.55 (s, 2H), 2.03 (s, 3H), 1.50 (dd, J=1.5, 7.2 Hz, 3H); MS for C17H20FN3O4 m/z 350.0 (M+H)⁺.

### Example 29

### Preparation of (S)—N-[3-(1-cyclopropylmethyl-7-fluoro-2-oxo-2,3-dihydro-1H-indol-5-yl)-2-oxo-oxazolidin-5-ylmethyl]-acetamide

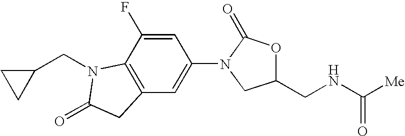

### Step 1: Preparation of 1-cyclopropylmethyl-7-fluoro-5-nitro-1,3-dihydro-indol-2-one

Crude (2,3-difluoro-5-nitrophenyl)acetic acid (Example 15, 2.50 g, 11.5 mmol) and (aminomethyl)cyclopropane (6 eq., 5.98 ml, 0.0691 mol) are mixed in DMSO (10 ml) and stirred at 45° C. for 20 hours. 2N Hydrochloric acid (40 ml) is added in one portion and the mixture stirred at room temperature for 2 hours. The resulting light yellow precipitate is filtered, washed with water and ether, and dried under vacuum to give the title compound as a yellow solid. HPLC r.t. 4.91 min; MS for C12H11FN2O3 m/z 251.2 (M+H)⁺.

### Step 2: Preparation of 5-amino-1-cyclopropylmethyl-7-fluoro-1,3-dihydro-indol-2-one

Iron powder (0.995 g, 18.1 mmol) is added in small portion to a mixture of 1-methyl-5-nitro-1,3-dihydro-indol-2-one (1.00 g, 4.52 mmol) and ammonium chloride (2.40 g, 45.2 mmol) in ethanol (20 ml) and water (10 ml) at 90° C. The reaction mixture is stirred vigorously and heated for 60 minutes, cooled to room temperature, and diluted with dichloromethane (100 ml). The mixture is filtered through celite, the organic layer separated and washed with water and brine, dried over sodium sulfate, and evaporated to give the title compound as a dark brown solid. HPLC r.t. 3.13 min; MS for C16H18FN3O3 m/z 221.0 (M+H)⁺.

### Step 3: Preparation of (R)-[3-(1-cyclopropylmethyl-7-fluoro-2-oxo-2,3-dihydro-1H-indol-5-ylamino)-2-hydroxy-propyl]-carbamic acid tert-butyl ester

5-Amino-1-cyclopropylmethyl-7-fluoro-1,3-dihydro-indol-2-one (Step 2, 1.00 g, 4.55 mmol), (S)-oxiranylmethyl-carbamic acid tert-butyl ester (0.79 g, 4.55 mmol) and lithium trifluoromethanesulfonate (0.618 g, 4.55 mmol) in acetonitrile (20 ml) are heated at 90° C. for 16 hours. The reaction mixture is diluted with ethyl acetate, washed with water and brine, dried (Na₂SO₄) and evaporated. Final purification by flash chromatography (70% ethyl acetate/hexane) gives the title compound as a white solid. HPLC r.t. 4.35 min; MS for C20H28FN3O4 m/z 394.1 (M+H)⁺.

### Step 4: Preparation of (S)-[3-(1-cyclopropylmethyl-7-fluoro-2-oxo-2,3-dihydro-1H-indol-5-yl)-2-oxo-oxazolidin-5-ylmethyl]-carbamic acid tert-butyl ester

Phosgene (20% solution in toluene, 1.50 ml, 14.1 mmol) is added to (R)-[3-(1-cyclopropylmethyl-7-fluoro-2-oxo-2,3-dihydro-1H-indol-5-ylamino)-2-hydroxy-propyl]-carbamic acid tert-butyl ester (0.74 g, 1.88 mmol) and triethylamine (0.95 ml, 9.41 mmol) in dichloromethane (15 ml) at 0° C. and then stirred at 0° C. for 20 minutes. The mixture is diluted with dichloromethane, washed with water and brine, dried (Na₂SO₄) and evaporated. The residue is purified by flash column chromatography (70% ethyl acetate/hexane) to give the title compound as a white solid. HPLC r.t. 5.39 min; MS for C21H26FN3O5 m/z 420.1 (M+H)⁺.

### Step 5: Preparation of (R)-5-(5-aminomethyl-2-oxo-oxazolidin-3-yl)-1-cyclopropylmethyl-7-fluoro-1,3-dihydro-indol-2-one

(S)-[3-(1-Cyclopropylmethyl-7-fluoro-2-oxo-2,3-dihydro-1H-indol-5-yl)-2-oxo-oxazolidin-5-ylmethyl]-carbamic acid tert-butyl ester (0.48 g, 1.14 mmol) is treated with 50% TFA/DCM (10 ml) for 60 minutes at room temperature. The reaction is evaporated and the title compound isolated as the TFA salt. HPLC r.t. 3.61 min; MS for C16H18FN3O3 m/z 320.3 (M+H)⁺.

### Step 6: Preparation of (S)—N-[3-(1-cyclopropylmethyl-7-fluoro-2-oxo-2,3-dihydro-1H-indol-5-yl)-2-oxo-oxazolidin-5-ylmethyl]-acetamide

Acetic anhydride (0.040 ml, 0.374 mmol) is added to (R)-5-(5-aminomethyl-2-oxo-oxazolidin-3-yl)-1-cyclopropylmethyl-7-fluoro-1,3-dihydro-indol-2-one (0.100 g, 0.312 mmol) and pyridine (0.080 ml, 0.938 mmol) in dichloromethane (15 ml) at 0° C. The reaction is stirred at 0° C. for 30 minutes and then allowed to warm at room temperature. The mixture is diluted with dichloromethane, washed with water and brine, dried (Na₂SO₄) and evaporated. The residue is purified by PTLC (10% MeOH/DCM) to give the title compound as an off white solid. HPLC r.t. 4.17 min; ¹H NMR (300 MHz, CDCl₃) 8.28 (t, 1H), 7.40 (s, 1H), 7.35 (s, 1H), 4.758-4.67 (m, 1H), 4.09 (t, J=9.0 Hz, 1H), 3.74 (dd, J=9, 6 Hz, 1H), 3.69 (s, 2H), 3.60 (d, J=7.2 Hz, 2H), 3.39 (t, J=5.4 Hz, 2H), 1.82 (s, 3H), 1.21-1.12 (m, 1H), 0.47-0.42 (m, 2H), 0.32-0.27 (m, 2H); MS for C18H20FN3O4 m/z 362.1 (M+H)⁺.

We claim:

1. A compound of formula I 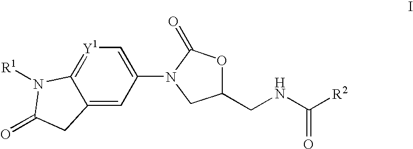
or a pharmaceutically acceptable salt thereof wherein:
Y¹ is CH or CF;
R¹ is —C₁₋₄alkyl, optionally substituted with a fluoro atom, or R¹ is —C₃₋₅cycloalkyl; and
R² is —C₁₋₂alkyl.

2. A compound of claim 1 wherein R² is CH₃.

3. A compound of claim 1 wherein R¹ is methyl, ethyl, propyl, or isopropyl.

4. A compound of claim 1 which is
(1) (S)—N-[3-(1-methyl-2-oxo-2,3-dihydro-1H-indol-5-yl)-2-oxo-oxazolidin-5-ylmethyl]-acetamide,
(2) (S)—N-[3-(1-methyl-2-oxo-2,3-dihydro-1H-indol-5-yl)-2-oxo-oxazolidin-5-ylmethyl]-propionamide,
(3) N-[3-(7-fluoro-1-methyl-2-oxo-2,3-dihydro-1H-indol-5-yl)-2-oxo-oxazolidin-5-ylmethyl]-acetamide,
(4) (S)—N-[3-(7-fluoro-1-methyl-2-oxo-2,3-dihydro-1H-indol-5-yl)-2-oxo-oxazolidin-5-ylmethyl]-propionamide,
(5) (S)—N-[3-(1-ethyl-2-oxo-2,3-dihydro-1H-indol-5-yl)-2-oxo-oxazolidin-5-ylmethyl]-acetamide,
(6) (S)—N-[3-(1-ethyl-2-oxo-2,3-dihydro-1H-indol-5-yl)-2-oxo-oxazolidin-5-ylmethyl]-propionamide,
(7) (S)—N-[3-(1-ethyl-7-fluoro-2-oxo-2,3-dihydro-1H-indol-5-yl)-2-oxo-oxazolidin-5-ylmethyl]-acetamide,
(8) (S)—N-[3-(1-ethyl-7-fluoro-2-oxo-2,3-dihydro-1H-indol-5-yl)-2-oxo-oxazolidin-5-ylmethyl]-propionamide,
(9) (S)—N-{3-[1-(2-fluoro-ethyl)-2-oxo-2,3-dihydro-1H-indol-5-yl]-2-oxo-oxazolidin-5-ylmethyl}-acetamide,
(10) (S)—N-{3-[1-(2-fluoro-ethyl)-2-oxo-2,3-dihydro-1H-indol-5-yl]-2-oxo-oxazolidin-5-ylmethyl}-propionamide,
(11) (S)—N-{3-[7-fluoro-1-(2-fluoro-ethyl)-2-oxo-2,3-dihydro-1H-indol-5-yl]-2-oxo-oxazolidin-5-ylmethyl}-acetamide,
(12) (S)—N-{3-[7-fluoro-1-(2-fluoro-ethyl)-2-oxo-2,3-dihydro-1H-indol-5-yl]-2-oxo-oxazolidin-5-ylmethyl}-propionamide,
(13) (S)—N-[3-(1-isopropyl-2-oxo-2,3-dihydro-1H-indol-5-yl)-2-oxo-oxazolidin-5-ylmethyl]-acetamide
(14) (S)—N-[3-(1-isopropyl-2-oxo-2,3-dihydro-1H-indol-5-yl)-2-oxo-oxazolidin-5-ylmethyl]-propionamide,
(15) (S)—N-[3-(7-fluoro-1-isopropyl-2-oxo-2,3-dihydro-1H-indol-5-yl)-2-oxo-oxazolidin-5-ylmethyl]-acetamide,
(16) (S)—N-[3-(7-fluoro-1-isopropyl-2-oxo-2,3-dihydro-1H-indol-5-yl)-2-oxo-oxazolidin-5-ylmethyl]-propionamide,
(17) (S)—N-[2-oxo-3-(2-oxo-1-propyl-2,3-dihydro-1H-indol-5-yl)-oxazolidin-5-ylmethyl]-acetamide,
(18) (S)—N-[2-oxo-3-(2-oxo-1-propyl-2,3-dihydro-1H-indol-5-yl)-oxazolidin-5-ylmethyl]-propionamide,
(19) (S)—N-[3-(7-fluoro-2-oxo-1-propyl-2,3-dihydro-1H-indol-5-yl)-2-oxo-oxazolidin-5-ylmethyl]-acetamide,
(20) (S)—N-[3-(7-Fluoro-2-oxo-1-propyl-2,3-dihydro-1H-indol-5-yl)-2-oxo-oxazolidin-5-ylmethyl]-propionamide,
(21) (S)—N-[3-(1-cyclopropyl-2-oxo-2,3-dihydro-1H-indol-5-yl)-2-oxo-oxazolidin-5-ylmethyl]-acetamide,
(22) (S)—N-[3-(1-cyclopropyl-2-oxo-2,3-dihydro-1H-indol-5-yl)-2-oxo-oxazolidin-5-ylmethyl]-propionamide,
(23) (S)—N-[3-(1-cyclopropyl-7-fluoro-2-oxo-2,3-dihydro-1H-indol-5-yl)-2-oxo-oxazolidin-5-ylmethyl]-acetamide,
(24) (S)—N-[3-(1-sec-butyl-2-oxo-2,3-dihydro-1H-indol-5-yl)-2-oxo-oxazolidin-5-ylmethyl]-acetamide,
(25) (S)—N-[3-(1-sec-butyl-2-oxo-2,3-dihydro-1H-indol-5-yl)-2-oxo-oxazolidin-5-ylmethyl]-propionamide,
(26) (S)—N-[3-(1-cyclopropylmethyl-2-oxo-2,3-dihydro-1H-indol-5-yl)-2-oxo-oxazolidin-5-ylmethyl]-acetamide,
(27) (S)—N-[3-(1-cyclopropylmethyl-2-oxo-2,3-dihydro-1H-indol-5-yl)-2-oxo-oxazolidin-5-ylmethyl]-propionamide,
(28) (S)—N-{3-[1-(2-fluoro-1-methyl-ethyl)-2-oxo-2,3-dihydro-1H-indol-5-yl]-2-oxo-oxazolidin-5-ylmethyl}-acetamide, or
(29) (S)—N-[3-(1-cyclopropylmethyl-7-fluoro-2-oxo-2,3-dihydro-1H-indol-5-yl)-2-oxo-oxazolidin-5-ylmethyl]-acetamide.

5. A pharmaceutical composition comprising a compound of formula I or a pharmaceutically acceptable salt thereof, and a pharmaceutically acceptable carrier.

6. A method for treating bacteria infections comprising administering to a mammal being treated a pharmaceutically effective amount of the compound of claim 1.

7. The method of claim 6 wherein the compound of claim 1 is administered orally.

8. The method of claim 6 wherein the compound of claim 1 is administered parenterally, topically, rectally, or intranasally.

9. The method of claim 6 wherein said compound is administered in an amount of from about 0.1 to about 100 mg/kg of body weight/day.

10. The method of claim 6 wherein said compound is administered in an amount of from about 1 to about 50 mg/kg of body weight/day.

11. The bacteria infection of claim 6 which is ear infections, eye infections, respiratory tract infections, skin and skin structure infections, bacterial endocarditis, osteomyelitis, endocarditis or diabetic foot.

12. The bacteria infection of claim 6 which is caused by gram-positive bacteria, gram negative bacteria, anaerobic organisms, and acid-fast organisms.

13. The bacteria infection of claim 6 which is caused by bacteria comprising staphylococci, streptococci, Enterococci, Haemophilus, Moraxella, bacteroides, clostridia, Mycobacteria, or Chlamydia.

14. The bacteria of claim 13 wherein staphylococci is S. aureus and S. epidennidis; wherein streptococci is S. pneumoniae of S. pyogenes; wherein Enterococci is E. faecalis; wherein Haemophilus is H. influenzae; wherein Moraxella is M. catarrhalis; and wherein Mycobacteria is M. tuberculosis; or Mycobacterium avium.

15. The bacteria infections of claim 6, which are infections caused by multi-drug resistant S. aureus.
